(19) United States
(12) Patent Application Publication
(10) Pub. No.: US 2003/0026792 A1
(43) Pub. Date: Feb. 6, 2003

(54) Adenovirus vectors specific for cells expressing carcinoembryonic antigen and methods of use thereof

(76) Inventors: Henry Lamparski, San Mateo, CA (US); Eric R. Schuur, Palo Alto, CA (US); Daniel R. Henderson, Palo Alto, CA (US)

Correspondence Address: Gladys H. Monroy, Morrison & Foerster LLP, 755 Page Mill Road, Palo Alto, CA, 94304-1018, US

(21) Appl. No.: 10/045,116

(22) Filed: Oct. 23, 2001

Related U.S. Application Data
(63) Continuation of application No. 09/033,555, filed on Mar. 2, 1998.
(60) Provisional application No. 60/039,763, filed on Mar. 3, 1997.

(51) Int. Cl. A61K 48/00; C12N 15/861

(52) U.S. Cl. 424/93.21; 435/456; 435/320.1

(57) ABSTRACT

Replication-competent adenovirus vectors specific for cells expressing carcinoembryonic antigen (CEA), and methods of use of such viruses are provided. These viruses comprise an adenoviral gene under control of a CEA transcriptional regulatory element (CEA-TRE). The gene can be, for example, a gene required for viral replication or the adenovirus death protein gene (ADP). The viruses can also comprise at least one other adenoviral gene under control of another transcriptional regulatory element specific to cells capable of which allow a CEA-TRE to function, such as a variant of a CEA-TRE. By providing for transcriptional initiating regulation dependent upon CEA expression, virus replication can be restricted to target cells which allow a CEA-TRE to function, such as cells expressing CEA, particularly carcinoma cells capable of expressing CEA. An adenovirus of the present invention can further comprise a heterologous gene such as a reporter gene under transcriptional control of a CEA-TRE. The adenovirus vectors can be used to detect and monitor samples for the presence of cells that allow a CEA-TRE to function, as well as to selectively kill malignant cells that allow a CEA-TRE to function.

### CROSS-REFERENCE TO RELATED APPLICATIONS

This application claims the benefit of U.S. Provisional Patent Application Serial No. 60/039,763, filed on Mar. 3, 1997.

### TECHNICAL FIELD

This invention relates to cell transfection using adenoviral vectors, especially replication-competent adenoviruses, and methods of their use. More specifically, it relates to cell-specific replication of adenovirus vectors in cells capable of expressing carcinoembryonic antigen (CEA), particularly CEA-associated tumor cells, through use of a carcinoembryonic antigen transcriptional regulatory element (CEA-TRE).

### BACKGROUND OF THE INVENTION

In spite of extensive medical research and numerous advances, cancer remains the second leading cause of death in the United States. Colorectal cancer is the third most common cancer and the second leading cause of cancer deaths. Lung cancer is one of the most refractory of solid tumors because inoperable cases are up to 60% and the 5-year survival is only 13%. In particular, adenocarcinomas, which comprise about one-half of the total lung cancer cases, are mostly chemo-radioresistant. Gastric carcinoma is one of the most prevalent forms of cancers in East Asia, including Japan and Korea. Although extensive surgical operations have been combined with chemotherapy and immunotherapy, the mortality of gastric cancer is still high, due to carcinomatous peritonitis and liver metastasis at advanced stages. Pancreatic cancer is virtually always fatal. Thus, current treatment prospects for many patients with these carcinomas are unsatisfactory, and the prognosis is poor.

Of particular interest is development of more specific, targeted forms of cancer therapy, especially in cancers that are difficult to treat successfully, such as hepatoma. In contrast to conventional cancer therapies, which result in relatively non-specific and often serious toxicity, more specific treatment modalities attempt to inhibit or kill malignant cells selectively while leaving healthy cells intact.

One possible treatment approach for cancers such as these carcinomas is gene therapy, whereby a gene of interest is introduced into the malignant cell. Boulikas (1997) Anticancer Res. 17:1471-1505. The gene of interest may encode a protein which converts into a toxic substance upon treatment with another compound, or an enzyme that converts a prodrug to an active drug. For example, introduction of the herpes simplex gene encoding thymidine kinase (HSV-tk) renders cells conditionally sensitive to ganciclovir (GCV). Zjilstra et al. (1989) Nature 342: 435; Mansour et al. (1988) Nature 336: 348; Johnson et al. (1989) Science 245: 1234; Adair et al. (1989) Proc. Natl. Acad Sci. USA 86: 4574; and Capecchi (1989) Science 244: 1288. Alternatively, the gene of interest may encode a compound that is directly toxic, such as diphtheria toxin (DT). For these treatments to be rendered specific to cancer cells, the gene of interest can be under control of a transcriptional regulatory element that is specifically (i.e., preferentially) increases transcription of an operably linked polynucleotide in the cancer cells. Cell- or tissue-specific expression can be achieved by using cell-specific enhancers and/or promoters. See generally Huber et al. (1995) Adv. Drug Delivery Rev. 17:279-292.

A variety of viral and non-viral (e.g., liposomes) vehicles, or vectors, have been developed to transfer these genes. Of the viruses, retroviruses, herpes virus, adeno-associated virus, Sindbis virus, poxvirus and adenoviruses have been proposed for use in gene transfer, with retrovirus vectors or adenovirus vectors being the focus of much current research. Verma and Somia (1997) Nature 389:239-242. Adenoviruses are among the most easily produced and purified, whereas retroviruses are unstable, difficult to produce and to purify, and may integrate into the host genome, raising the possibility of dangerous mutations. Moreover, adenovirus has the advantage of effecting high efficiency of transduction and does not require cell proliferation for efficient cell transduction. For general background references regarding adenovirus and development of adenoviral vector systems, see Graham et al. (1973) Virology 52:456-467; Takiffet al. (1981) Lancet 11:832-834; Berkner et al. (1983) Nucleic Acid Research 11: 6003-6020; Graham (1984) EMBO J 3:2917-2922; Bett et al. (1993) J. Virology 67:5911-5921; and Bett et al. (1994) Proc. Natl. Acad. Sci. USA 91:8802-8806.

When used as gene transfer vehicles, adenovirus vectors are often designed to be replication-defective and are thus deliberately engineered to fail to replicate in the target cells of interest. In these vehicles, the early adenovirus gene products E1A and/or E1B are deleted and provided in trans by the packaging cell line 293. Graham et al. (1987) J. Gen. Virol 36:59-72; Graham (1977) J. Genetic Virology 68:937-940. The gene to be transduced is commonly inserted into adenovirus in the deleted E1A and/or E1B region of the virus genome. Bett et al. (1994). Replication-defective adenovirus vectors as vehicles for efficient transduction of genes have been described by, inter alia, Stratford-Perricaudet (1990) Human Gene Therapy 1:241-256; Rosenfeld (1991) Science 252:431-434; Wang et al. (1991) Adv. Exp. Med. Biol. 309:61-66; Jaffe et al. (1992) Nat. Gent. 1:372-378; Quantin et al. (1992) Proc. Natl. Acad. Sci. USA 89:2581-2584; Rosenfeld et al. (1992) Cell 68:143-155; Stratford-Perricaudet et al. (1992) J. Clin. Invest. 90:626-630; Le Gal Le Salle et al. (1993) Science 259:988-990 Mastrangeli et al. (1993) J. Clin. Invest. 91:225-234; Ragot et al. (1993) Nature 361:647-650; Hayaski et al. (1994) J. Biol. Chem. 269:23872-23875; and Bett et al. (1994).

The virtually exclusive focus in the development of adenoviral vectors for gene therapy is use of adenovirus merely as a vehicle for introducing the gene of interest, not as an effector in itself. Replication of adenovirus has been viewed as an undesirable result, largely due to the host immune response. In the treatment of cancer by replication-defective adenoviruses, the host immune response limits the duration of repeat doses at two levels. First, the capsid proteins of the adenovirus delivery vehicle itself are immunogenic. Second, viral late genes are frequently expressed in transduced cells, eliciting cellular immunity. Thus, the ability to repeatedly administer cytokines, tumor suppressor genes, ribozymes, suicide genes, or genes which convert a prodrug to an active drug has been limited by the immunogenicity of both the gene transfer vehicle and the viral gene products of the transfer vehicle as well as the transient nature of gene expression. There is a need for vector constructs that are capable of eliminating essentially all cancerous cells in a minimum number of administrations before specific immunological response against the vector prevents further treatment.

A completely separate and unrelated area of research pertains to the description of tissue-specific transcriptional regulatory proteins.

Carcinoembryonic antigen (CEA)

Carcinoembryonic antigen (CEA) is a 180-kiloDalton (kDa) glycosylated tumor-associated antigen present on endodermally-derived neoplasms of the gastrointestinal tract, such as colorectal, gastric (stomach) and pancreatic cancer, as well as other adenocarcinomas such as breast and lung cancers. CEA is of clinical interest because circulating CEA can be detected in the great majority of patients with CEA-positive tumors. In lung cancer, about 50% of total cases have circulating CEA, with high concentrations of CEA (greater than 20 ng/ml) often detected in adenocarcinomas. Approximately 50% of patients with gastric carcinoma are serologically positive for CEA.

The 5′ upstream flanking sequence of the human CEA gene has been isolated and characterized. Willcocks et al. (1990) Genomics 8:492-500; Richards et al. (1993) DNA Seq. 4:185-196; Richards et al. (1995) Human Gene Ther. 6:881-893; Hauck et al. (1995) J. Biol. Chem. 270:3602-3610; WO/95/14100. The CEA promoter has been shown to confer cell-specific activity. Schrewe et al. (1990) Mol. Cell. Biol. 10:2738-2748. In addition, cell-specific enhancers have been found. WO/95/14100. An enhancer is a cis-acting transcriptional regulatory element known to play a major role in determination of cell specificity of gene expression. The enhancer is also typically characterized by its ability to augment transcription over a long distance and relatively independently of orientation and position with respect to its respective gene. A promoter is located immediately 5′ (upstream) of the transcription start site and generally includes an AT-rich region called a TATA box.

Several approaches for gene therapy using the cell-specific CEA promoter/enhancer to treat CEA-associated cancers have been described. Richards et al. (1995) reported the generation of stable cell lines in which CEA transcriptional regulatory sequences were used to regulate the expression of cytosine deaminase (CD) in colorectal cancer. Treatment of mouse xenografts containing the CEA-CD gene with 5-FC resulted in significant tumor effects in vivo. The CEA/CD chimeric gene for tumor-specific suicide gene therapy of CEA-positive tumors is described in patent application WO 95/14100. Osaki et al. (1994) describe adenovirus-mediated prodrug gene therapy in which the CEA promoter was used to restrict HSV-tk expression to CEA-producing human lung cancer cells, rendering them sensitive to the nucleoside analog GCV. Cancer Res. 54:5258-5261. Similarly, Tanaka et al. (1996) describe using the CEA promoter to drive selectively expression of HSV-tk, which conferred GCV sensitivity to gastric cancer cells. Cancer Res. 56:1341-1345. In all of these publications, the adenovirus constructs are replication defective, and the entire focus in experimental approach is using the CEA 5′ upstream transcriptional regulatory region(s) to control expression of a non-adenovirus gene. Replication-deficient adenovirus is viewed as a therapeutic gene delivery vehicle, not as an agent per se for effecting selective growth inhibition.

Carcinomas of the gastrointestinal tract are often not curable by standard therapies. Thus, it is critical to develop new therapeutic approaches for these diseases. The present invention addresses this need by providing adenoviral vectors specific for replication in CEA-producing cells.

All publications cited herein are hereby incorporated by reference in their entirety.

### SUMMARY OF THE INVENTION

In one embodiment, the invention provides an adenovirus vector comprising an adenovirus gene under transcriptional control of a carcinoembryonic antigen transcriptional regulatory element (CEA-TRE). A CEA-TRE is capable of mediating gene expression specific to cells capable of expressing CEA or capable of CEA-TRE-mediated transcription. The CEA-TRE can comprise a promoter and/or enhancer from a carcinoembryonic antigen gene, provided that the CEA-TRE is capable of mediating gene expression specific to cells capable of expressing CEA. In one embodiment, a CEA-TRE comprises a promoter from a carcinoembryonic antigen gene. In one embodiment, a CEA-TRE comprises an enhancer from a carcinoembryonic antigen gene. In one embodiment, a CEA-TRE comprises a promoter from a carcinoembryonic antigen gene and an enhancer from a carcinoembryonic antigen gene.

In certain embodiments, the invention provides an adenovirus vector comprising an adenovirus gene under transcriptional control of a CEA-TRE. In one embodiment, a CEA-TRE is human. In one embodiment, a CEA-TRE comprises a CEA-specific promoter and enhancer.

In one embodiment, the CEA-TRE comprises an approximately 0.5 kb promoter segment (about −402 to about +69 relative to the transcriptional start, SEQ ID NO:1), which is specific for cells that allow a CEA-TRE to function, such as CEA-producing cells. Accordingly, the invention also includes an adenovirus vector in which the CEA-TRE comprises SEQ ID NO: 1. In another embodiment, the CEA-TRE comprises the sequence from about −299 to about +69 (nucleotides about 104 to about 472 of SEQ ID NO: 1). In another embodiment, the CEA-TRE comprises the sequence from about −90 to about +69 (nucleotides about 313 to about 472 of SEQ ID NO: 1). In various embodiments, the adenovirus vector comprises any of the sequences listed above and further comprises the region from about −14 kb to about −10.6 kb, about −13.6 kb to about −10.6 kb, or −6.1 kb to about −3.8 kb relative to the transcriptional start.

In some embodiments, the adenovirus gene under transcriptional control of a CEA-TRE contributes to cytotoxicity (directly or indirectly), such as a gene essential for viral replication. In one embodiment, the replication gene is an early gene. In another embodiment, the early gene is E1A. In another embodiment, the early gene is E1B. In yet another embodiment, both E1A and E1B are under transcriptional control of a CEA-TRE. In other embodiments, the adenovirus gene essential for replication is a late gene. In various embodiments, the additional late gene is L1, L2, L3, L4, or L5.

In another embodiment, the adenovirus gene under control of a CEA-TRE is the adenovirus death protein (ADP).

In another embodiment, the adenovirus comprising an adenovirus gene under transcriptional control of a CEA-TRE further comprises at least one additional adenovirus gene under transcriptional control of at least one other transcriptional element specific to a cell allowing function of a CEA-TRE.

In one embodiment, the at least one other transcriptional element specific to CEA-expressing cells is another copy of a CEA-TRE. In one embodiment, the at least one other transcriptional element specific to CEA-expressing cells is a variant of a CEA-TRE which is different from the first CEA-TRE.

In other embodiments, the adenovirus vector can further comprise a heterologous gene under transcriptional control of a CEA-TRE. In one embodiment, the heterologous gene is a reporter gene. In one embodiment, the heterologous gene is conditionally required for cell survival.

In one embodiment, a composition comprises any adenovirus disclosed herein. In one embodiment, this composition comprises a pharmaceutically acceptable excipient.

In another aspect, the invention provides pharmaceutical compositions comprising an effective amount of an adenovirus vector(s) described herein.

In another aspect, the invention provides kits which contain an adenoviral vector(s) described herein.

In another aspect, the invention provides a host cell transformed with any adenovirus vector(s) described herein.

In another embodiment, a method of treating a CEA-associated tumor in an individual is provided, the method comprising the step of administering to the individual an effective amount of an adenovirus vector in which an adenovirus gene is under transcriptional control of a CEA-TRE. In one embodiment, a CEA-associated tumor comprises cells capable of expressing CEA. In one embodiment, the adenovirus gene is essential for viral replication. In one embodiment, the adenovirus gene is an early gene. In one embodiment, the adenovirus gene is E1A. In one embodiment, the adenovirus gene is E1B. In one embodiment, the adenovirus gene is a late gene. In one embodiment, the adenovirus gene is ADP. In one embodiment, the CEA-TRE comprises an enhancer from a CEA gene. In one embodiment, the CEA-TRE comprises a promoter from a CEA gene. In one embodiment, the CEA-TRE comprises a promoter from a CEA gene and an enhancer from a CEA gene. In one embodiment, the adenovirus further comprises at least one additional adenovirus gene under transcriptional control of at least one additional CEA-TRE. In one embodiment, the at least one additional CEA-TRE is different from the first CEA-TRE. In one embodiment, the at least one additional adenovirus gene is essential for viral replication. In one embodiment, the at least one additional adenovirus gene is an early gene. In one embodiment, the at least one additional adenovirus gene is an early gene. In one embodiment, the at least one additional adenovirus gene is E1A. In one embodiment, the at least one additional adenovirus gene is E1B. In one embodiment, the at least one additional adenovirus gene is a late gene. In various embodiments, the late gene can be L1, L2, L3, L4, or L5. In one embodiment, the at least one additional adenovirus gene is ADP.

Another embodiment of the invention is an adenovirus which replicates preferentially in mammalian cells that allow a CEA-TRE to function.

In another aspect, methods are provided for propagating an adenovirus specific for cells that allow a CEA-TRE to function, said method comprising combining an adenovirus vector(s) described herein with cells that allow a CEA-TRE to function, whereby said adenovirus is propagated.

In another aspect, methods are provided for detecting cells that allow a CEA-TRE to function in a biological sample, comprising contacting a biological sample with an adenovirus vector(s) described herein, under conditions that allow a CEA-TRE to function in cells that allow a CEA-TRE to function; and determining if CEA-TRE mediates gene expression in the biological sample, wherein CEA-TRE-mediated gene expression is indicative of the presence of cells that allow a CEA-TRE to function.

In one embodiment, a method is provided for detecting cells that allow a CEA-TRE to function in a biological sample, the method comprising the steps of: contacting a biological sample with an adenovirus comprising a gene under transcriptional control of a CEA-TRE, under conditions suitable for CEA-TRE mediated gene expression in cells that allow a CEA-TRE to function; and determining if the CEA-TRE mediates gene expression in the biological sample, where CEA-TRE-mediated gene expression is indicative of the presence of cells that allow a CEA-TRE to function. In one embodiment, the gene is a heterologous (non-adenovirus) gene. In one embodiment, the gene is a reporter gene, and production of the product of the reporter gene is detected.

In another aspect, methods are provided for conferring selective cytotoxicity on a target cell, said method comprising contacting a cell which allows a CEA-TRE to function with any adenovirus vector(s) described herein, wherein the adenovirus enters the cell.

In one embodiment, an adenovirus is provided which further comprises a heterologous gene under transcriptional control of a CEA-TRE. In one embodiment, the heterologous gene is a reporter gene. In one embodiment, the heterologous gene is conditionally required for cell survival. In one embodiment, a method is provided for detecting cells that allow a CEA-TRE to function in a sample comprising the steps of: contacting a biological sample with an adenovirus vector comprising a heterologous gene under transcriptional control of a CEA-TRE, under conditions suitable for CEA-TRE-mediated gene expression in cells that allow a CEA-TRE to function; and determining if the CEA-TRE mediates gene expression in the biological sample, where CEA-TRE-mediated gene expression is indicative of the presence of cells that allow a CEA-TRE to function.

In one embodiment, a method is provided for modifying the genotype of a target cell, the method comprising contacting a cell that allows a CEA-TRE to function with any adenovirus vector(s) described herein, wherein the vector enters the cell.

In one embodiment, a method for conferring selective cytotoxicity on a target cell is provided, the method comprising contacting a cell that allows a CEA-TRE to function with any adenovirus vector(s) described herein, wherein the vector enters the cell.

### BRIEF DESCRIPTION OF THE DRAWINGS

FIG. 1 depicts a restriction map of the 5′ flanking region of the human CEA gene.

FIG. 2 depicts the nucleotide sequence of the 5′ flanking region of CEA (to about +537).

FIG. 3 is a schematic depicting various adenoviral vector constructs as described in Example 1.

FIG. 4 is a schematic depiction of an adenoviral vector in which E1A and E1B are under control of a CEA-TRE, with E1A and E1B in opposite orientations.

FIGS. 5A and B are schematic depictions an adenovirus death protein (ADP) cassette for insertion into Ad. Arrows underneath FIG. 5A indicate positions of primers. FIG. 5B depicts the annealed fragment containing the Y leader sequence and the ADP coding sequence.

FIG. 6 is a graph depicting cytotoxicity of an adenoviral vector containing the coding sequence for adenoviral death protein (ADP), CN751 (solid squares), compared to control CN702 (solid circles), Rec 700 (solid triangles) and mock infection (Xs).

FIG. 7 is a graph comparing extracellular virus yield of CN751 (solid squares) and CN702 (solid circles).

FIG. 8 is a graph comparing tumor volume in mice harboring LNCaP tumor xenografts challenged with CN751 (“H”), CN702 (“J”), or buffer (“B”).

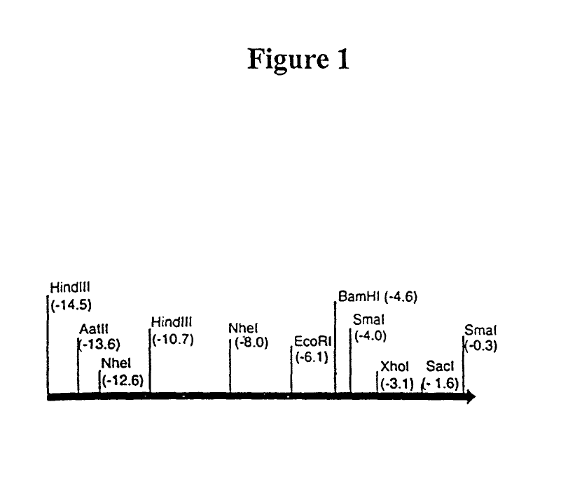

### MODES FOR CARRYING OUT THE INVENTION

We have discovered and constructed replication-competent adenovirus vectors containing a carcinoembryonic antigen transcriptional regulatory element (CEA-TRE) which can preferentially replicate in cells capable of expressing carcinoembryonic antigen (CEA) and have developed methods using these adenovirus vectors. The adenovirus vectors of this invention comprise at least one adenovirus gene under the transcriptional control of a CEA-TRE. The adenovirus gene can be, for example, a gene that contributes to cytotoxicity (directly or indirectly), such as a gene necessary for adenoviral replication. This replication gene is preferably at least one early gene. Alternatively, the adenovirus gene under control of a CEA-TRE can be an adenovirus death protein (ADP) gene. Alternatively, the adenovirus gene under control of a CEA-TRE can be a late replication gene. The adenovirus can optionally comprise at least one other gene such as an adenovirus gene or transgene under control of another TRE which is different from the CEA-TRE. By providing for cell-specific transcription of at least one adenovirus gene required for replication, the invention provides adenovirus vectors that can be used for specific cytotoxic effects due to selective replication. Selective replication is especially useful in the cancer context, in which targeted cell killing is desirable. The adenovirus vectors of this invention are useful for treatment of CEA-associated tumors, such as colorectal carcinomas. The vectors can also be useful for detecting the presence of cells that allow a CEA-TRE to function in, for example, an appropriate biological (such as clinical) sample. Further, the adenovirus vector(s) can optionally selectively produce one or more proteins of interest in a target cell by using a CEA-TRE.

We have found that the adenovirus vectors of the invention replicate preferentially in cells that allow a CEA-TRE to function, such as cells expressing CEA (i.e., at a significantly higher yield). This replication preference is indicated by comparing the level of replication (i.e., titer) in cells that allow a CEA-TRE to function to the level of replication in cells that do not allow a CEA-TRE to function. The replication preference is even more significant, as the adenovirus vectors of the invention actually replicate at a significantly lower rate in cells that do not allow a CEA-TRE to function than wild-type cells. Comparison of the titer of a cell type that allows a CEA to function to the titer of a cell type that does not allow a CEA-TRE to function provides a key indication that the overall replication preference is enhanced due to depressed replication in cells that do not allow a CEA-TRE to function as well as the replication in cells that allow a CEA-TRE to function. Thus, the invention uses and takes advantage of what has been considered an undesirable aspect of adenoviral vectors, namely, their replication and possibly concomitant immunogenicity. Runaway infection is prevented due to the cell-specific requirements for viral replication. Without wishing to be bound by any particular theory, the inventors note that production of adenovirus proteins can serve to activate and/or stimulate the immune system, either generally and/or specifically toward the target cells producing adenoviral proteins, which can be an important consideration in the cancer context, where patients are often moderately to severely immunocompromised.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Wei & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

For techniques related to adenovirus, see, inter alia, Felgner and Ringold (1989) Nature 337:387-388; Berkner and Sharp (1983) Nucl. Acids Res. 11:6003-6020; Graham (1984) EMBO J. 3:2917-2922; Bett et al. (1993) J. Virology 67:5911-5921; and Bett et al. (1994) Proc. Natl. Acad. Sci. USA 91:8802-8806.

Definitions

As used herein, a “carcinoembryonic antigen transcriptional response element”, or “CEA-TRE” is polynucleotide sequence, preferably a DNA sequence, which increases transcription of an operably linked polynucleotide sequence in a host cell that allows a CEA-TRE to function, such as a cell that expresses CEA. The CEA-TRE is responsive to transcription factors and/or co-factor(s) associated with CEA-producing cells and comprises at least a portion of the CEA promoter and/or enhancer. A CEA-TRE can also include other sequences, such as other transcription control sequences, preferably a CEA enhancer. Methods are described herein for measuring the activity of a CEA-TRE and thus for determining whether a given cell allows a CEA-TRE to function.

As described in more detail herein, a CEA-TRE can comprise any number of configurations, including, but not limited to, a CEA promoter; a CEA enhancer; any transcriptional regulatory elements of a CEA promoter and/or enhancer capable of mediating transcription specifically in CEA-expressing cells; a CEA promoter and a CEA enhancer; a CEA promoter and a heterologous (non-CEA) enhancer; a heterologous promoter and a CEA enhancer; and multimers of the foregoing. The promoter and enhancer of a CEA-TRE may be in any orientation and/or distance from the coding sequence of interest, as long as the desired CEA cell-specific transcriptional activity is obtained. Transcriptional activation can be measured in a number of ways known in the art (and described in more detail below), but is generally measured by detection and/or quantitation of the protein product of the coding sequence under control of (i.e., operatively linked to) the CEA-TRE. As discussed herein, CEA-TRE can be of varying lengths, and of varying sequence composition. By “transcriptional activation” or “increase in transcription,” it is intended that transcription is increased above basal levels in the target cell (i.e., a cell allowing a CEA-TRE to function) by at least about 2-fold, preferably at least about 5-fold, preferably at least about 10-fold, more preferably at least about 20-fold, more preferably at least about 50-fold, more preferably at least about 100-fold, more preferably at least about 200-fold, even more preferably at least about 400-fold to about 500-fold, even more preferably at least about 1000-fold. Basal levels are generally the level of activity (if any) in a cell that does not allow a CEA-TRE to function, or the level of activity (if any) of a reporter construct lacking a CEA-TRE as tested in a cell that allows a CEA-TRE to function.

A “functionally-preserved” variant of a CEA-TRE is a CEA-TRE which differs from another CEA-TRE, but still retains the ability to increase transcription of an operably linked polynucleotide, especially cell-specific transcription activity. The difference in a CEA-TRE can be due to differences in linear sequence, arising from, for example, single or multiple base mutation(s), addition(s), deletion(s), insertion(s), and/or modification(s) of the bases. The difference can also arise from changes in the sugar(s), and/or linkage(s) between the bases of a CEA-TRE.

As used herein, “expression in cells that allow a CEA-TRE to function”, “expression specific to cells capable of expressing CEA,” and the like indicate gene expression which occurs primarily in cells containing all the transcriptional factor(s) and/or co-factor(s) needed to mediate transcription from a CEA-TRE, but to a lesser degree in other cells. These terms (as well as the terms “transcriptional activation” or an “increase in transcription”) indicate that this gene expression is at least about 2-fold, preferably at least about 5-fold, preferably at least about 10-fold, more preferably at least about 20-fold, more preferably at least about 50-fold, more preferably at least about 100-fold, more preferably at least about 200-fold, even more preferably at least about 400-to about 500-fold, even more preferably at least about 1000-fold, greater in cells that allow a CEA-TRE to function or in cells expressing gene products necessary for expressing CEA. Methods of measuring levels (whether relative or absolute) of the expression are known in the art or described here. Genes demonstrating gene expression specific to cells that allow a CEA-TRE to function include, but are not limited to, factors required for transcription from a CEA-TRE. Such factors are typically produced by CEA-producing cells such as colonic tumor tissue cells, aberrant crypt foci, and other CEA-producing cells or CEA-associated tumor cells such as SW403, SW1463, SW837, NCIH508, LoVo, MKN1, MKN28, MKN45, and A549.

A “CEA-TRE different from another CEA-TRE” indicates that the two CEA-TREs have nucleotide sequence dissimilarity.

An “adenovirus vector” or “adenoviral vector” (used interchangeably) is a term well understood in the art and generally comprises a polynucleotide (defined below) comprising all or a portion of an adenovirus genome. For the purposes of the present invention, an adenovirus vector contains a CEA-TRE operably linked to a polynucleotide. The operably linked polynucleotide can be adenoviral or heterologous. An adenoviral vector construct of the present invention can be in any of several forms, including, but not limited to, naked DNA. DNA encapsulated in an adenovirus coat, DNA packaged in another viral or viral-like form (such as herpes simplex virus and AAV), DNA encapsulated in liposomes, DNA complexed with polylysine, complexed with synthetic polycationic molecules, conjugated with transferrin, and complexed with compounds such as PEG to immunologically “mask” the molecule and/or increase half-life, and conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides. For purposes of this invention, adenovirus vectors are replication-competent in a target cell.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P-NH₂) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothiate linkage can be used in place of a phosphodiester linkage. Braun et al. (1988) J. Immunol. 141: 2084-9; Latimer et al. (1995) Mol. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

One of skill in the art would recognize that point mutations and deletions can be made to a CEA-TRE disclosed herein without altering the ability of the sequence to increase transcription. Preferably, the polynucleotide of the present invention is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A polynucleotide or polynucleotide region has a certain percentage (for example, 80%, 85%, 90%, or 95%) of “sequence identity” to another sequence means that, when aligned, that percentage of bases are the same in comparing the two sequences. This alignment and the percent homology or sequence identity can be determined using software programs known in the art, for example, those described in Current Protocols in Molecular Biology (Ausubel et al., eds., 1987), Supp. 30, section 7.7.18, Table 7.7.1. A preferred alignment program is ALIGN Plus (Scientific and Educational Software, Pennsylvania).

“Under transcriptional control” is a term well-understood in the art and indicates that transcription of a polynucleotide sequence, usually a DNA sequence, depends on its being operably (operatively) linked to an element which contributes to the initiation of, or promotes, transcription. As defined herein, “operably linked” refers to a juxtaposition wherein the elements are in an arrangement allowing them to function.

As used herein, “cytotoxicity” is a term well understood in the art and refers to a state in which one or more of a cell's usual biochemical or biological functions are aberrantly compromised (i.e., inhibited or elevated). These activities include, but are not limited to, metabolism; cellular replication; DNA replication; transcription; translation; and uptake of molecules. “Cytotoxicity” includes cell death and/or cytolysis. Assays are known in the art which indicate cytotoxicity, such as dye exclusion, ³H-thymidine uptake, and plaque assays. The term “selective cytotoxicity”, as used herein, refers to the cytotoxicity conferred by an adenovirus vector of the present invention on a cell which allows a CEA-TRE to function when compared to the cytotoxicity conferred by the adenovirus on a cell which does not allow a CEA-TRE to function. Such cytotoxicity may be measured, for example by plaque assays, by reduction or stabilization in size of a tumor comprising target cells, or the reduction or stabilization of serum levels of a marker characteristic of the tumor cells or a tissue-specific marker, e.g., a cancer marker such as CEA or PSA.

“Replication” and “propagation” are used interchangeably and refer to the ability of an adenovirus vector of the invention to reproduce, or proliferate. This term is well understood in the art. For purposes of this invention, replication involves production of adenovirus proteins and is generally directed to reproduction of adenovirus. Replication can be measured using assays standard in the art and described herein, such as a burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

A “heterologous gene” or “transgene” is any gene that is not present in wild-type adenovirus. Preferably, the transgene will also not be expressed or present in the target cell, prior to introduction by the adenovirus vector. Examples of preferred transgenes are provided below.

A “heterologous” promoter or enhancer is one which is not associated with or derived from a CEA 5′ flanking sequence. Examples of a heterologous promoter or enhancer are the albumin promoter or enhancer and other viral promoters and enhancers, such as from SV40.

An “endogenous” promoter, enhancer, or TRE is native to or derived from adenovirus.

A “host cell” includes an individual cell or cell culture which can be or has been a recipient of any vector(s) of this invention. Host cells include progeny of a single host cell, and the progeny may not necessarily be completely identical (in morphology or of total DNA complement) to the original parent cell due to natural, accidental, or deliberate mutation and/or change. A host cell includes cells transfected or infected in vivo with a vector of this invention.

A “target cell” is any cell that allows a CEA-TRE to function. Preferably, a target cell is a mammalian cell, preferably a cell that allows a CEA-TRE to function, more preferably a human cell that allows a CEA-TRE to function, such as a cell that expresses CEA.

As used herein, “neoplastic cells” and “neoplasia” refers to cells which exhibit relatively autonomous growth, so that they exhibit an aberrant growth phenotype characterized by a significant loss of control of cell proliferation. Neoplastic cells can be benign or malignant.

A “cell which allows a CEA-TRE to function,” a “cell capable of expressing CEA” or the like is a cell in which a CEA-TRE, when operably linked to, for example, a reporter gene, increases expression of the reporter gene at least about 2-fold, preferably at least about 5-fold, preferably at least about 10-fold, more preferably at least about 20-fold, more preferably at least about 50-fold, more preferably at least about 100-fold, more preferably at least about 200-fold, even more preferably at least about 400- to 500-fold, even more preferably at least about 1000-fold, when compared to the expression of the same reporter gene when not linked to the CEA-TRE. Methods for measuring levels (whether relative or absolute) of expression are known in the art and are described herein. In contrast, cells that do not allow a CEA-TRE to function indicate cells that are not known to produce, or are not capable of producing, detectable levels of CEA. However, as more sensitive detection methods are developed, some or all of these cells may be found to produce hitherto undetectably low levels or unstable forms of CEA. Cells that do not allow a CEA-TRE to function are generally incapable or only very poorly capable of mediating CEA-TRE-mediated gene expression and include LNCaP, HBL-100, HLF, HLE, 3T3, Hep3B, HuH7, CADO-LC9, and HeLa cells.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

An “individual” is a vertebrate, preferably a mammal more preferably a human. Mammals include, but are not limited to, farm animals, sport animals, and pets.

An “effective amount” is an amount sufficient to effect beneficial or desired clinical results. An effective amount can be administered in one or more administrations. For purposes of this invention, an effective amount of an adenoviral vector is an amount that is sufficient to palliate ameliorate, stabilize, reverse, slow or delay the progression of the disease state.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease means that the extent and/or undesirable clinical manifestations of a disease state are lessened and/or time course of the progression is slowed or lengthened, as compared to not administering adenoviral vectors of the present invention.

Adenoviral vectors having replication specificity for cells that allow a CEA-TRE to function

The present invention also provides adenoviral vector constructs which comprise an adenoviral gene under transcriptional control of a CEA-TRE. Preferably, the adenoviral gene is one that contributes to cytotoxicity (whether directly and/or indirectly), more preferably one that contributes to or causes cell death and even more preferably, the adenoviral gene under transcriptional control of a CEA-TRE is a gene essential for adenoviral replication. Examples of an adenoviral gene that contributes to cytotoxicity include, but are not limited to, an adenoviral death protein (ADP). When the adenovirus vector(s) is selectively (i.e., preferentially) replication-competent for propagation in target cells that allow a CEA-TRE to function, these cells will be preferentially killed upon adenoviral proliferation. By combining the adenovirus vector(s) with the mixture of (e.g., CEA-producing) malignant and normal cells, for example, in vitro or in vivo, the adenovirus vector(s) preferentially replicate in the target malignant cells. Once the target cells are destroyed due to selective cytotoxic and/or cytolytic replication, the adenovirus vector replication is significantly reduced, thus lessening the probability of runaway infection and undesirable bystander effects. In vitro cultures may be retained to continually monitor the mixture (such as, for example, a biopsy or other appropriate biological sample) for occurrence (i.e., presence) and/or recurrence of the target cell, e.g., a CEA-producing neoplastic cell. To ensure cytotoxicity further, one or more transgenes having a cytotoxic effect may also be present and under selective transcriptional control. In this embodiment, one may provide higher confidence that the target cells will be destroyed. Additionally, or alternatively, an adenovirus gene that contributes to cytotoxicity and/or cell death (such as ADP) may be included in the adenoviral vector, either free of, or under, selective transcriptional control.

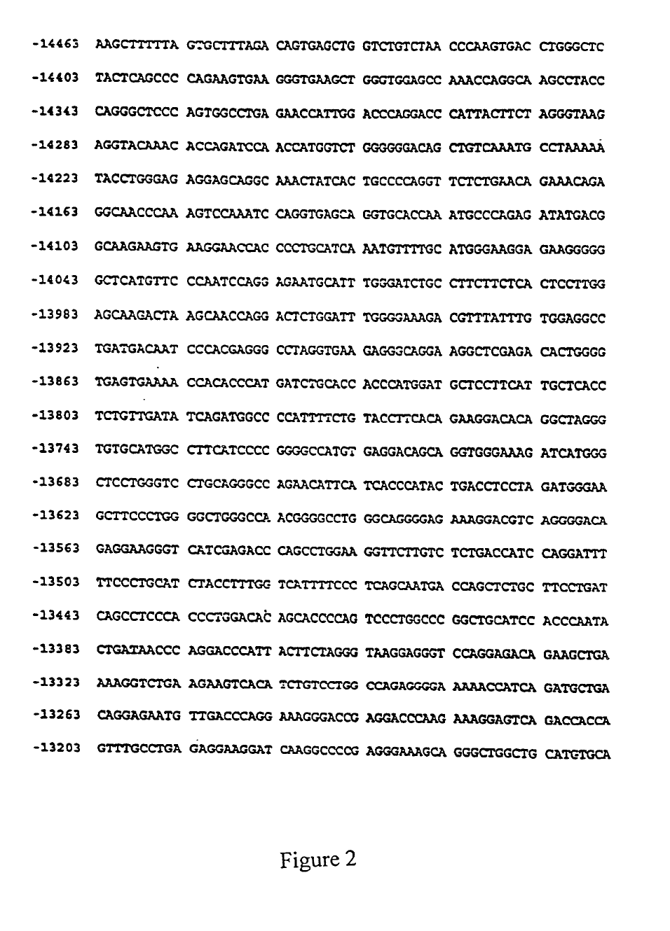

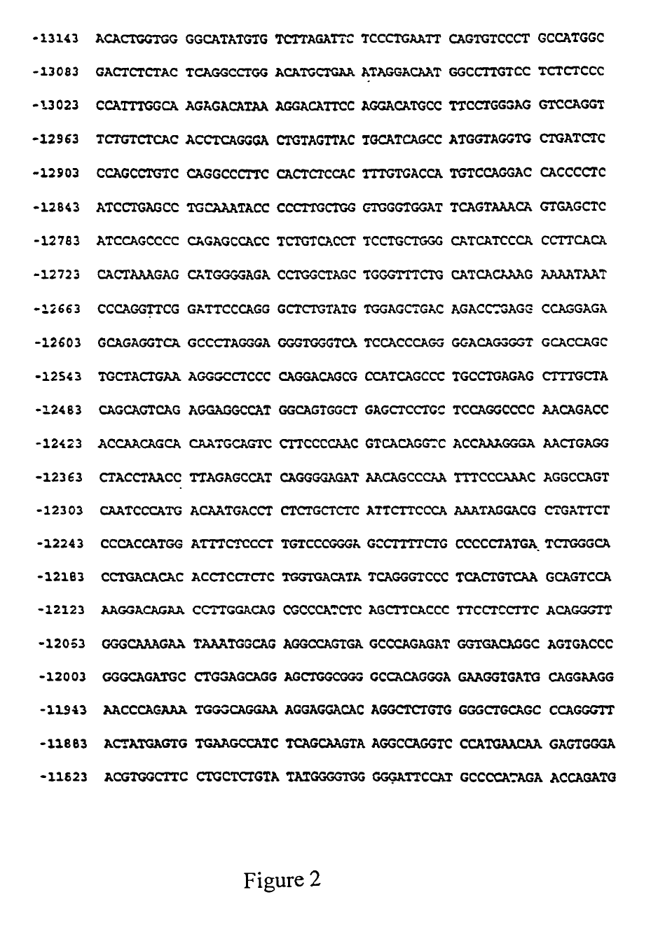

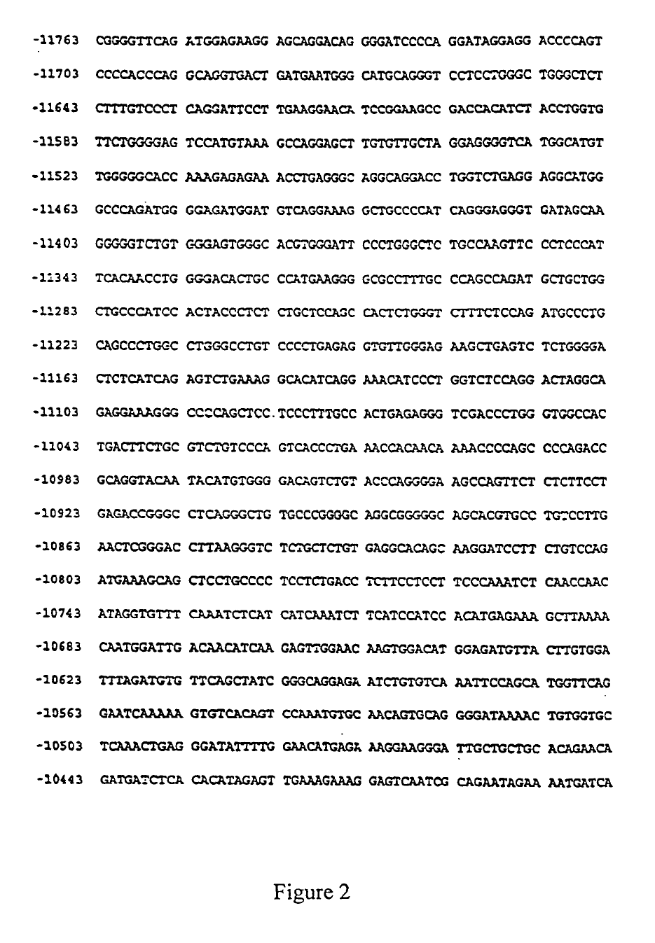

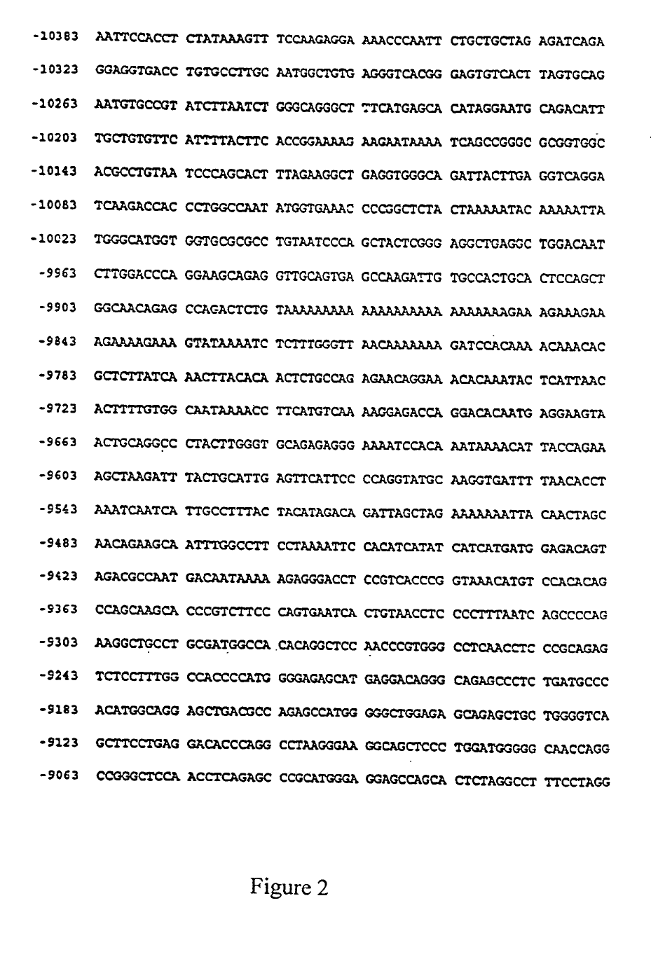

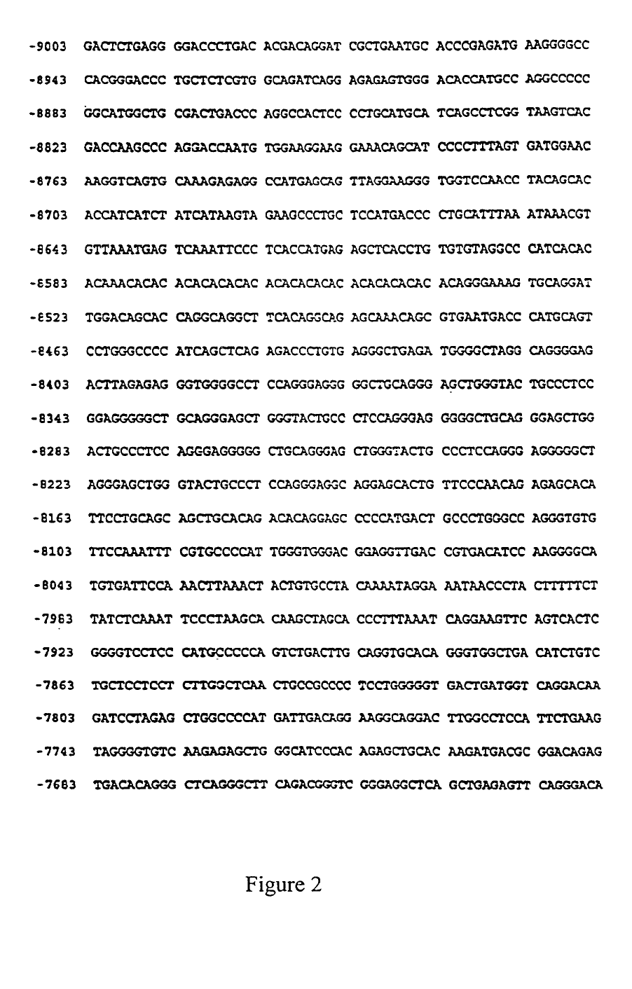

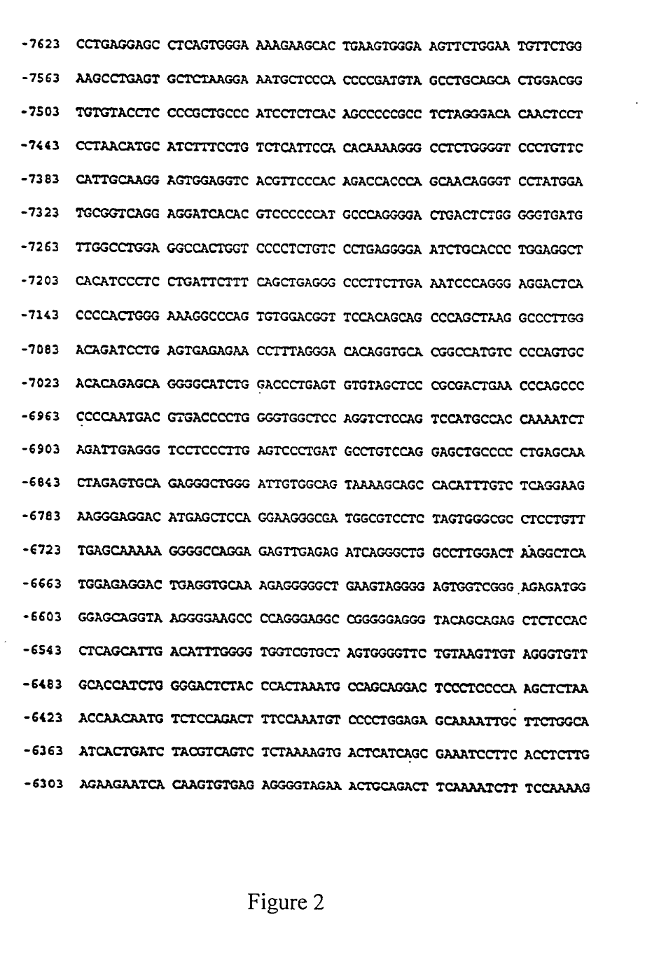

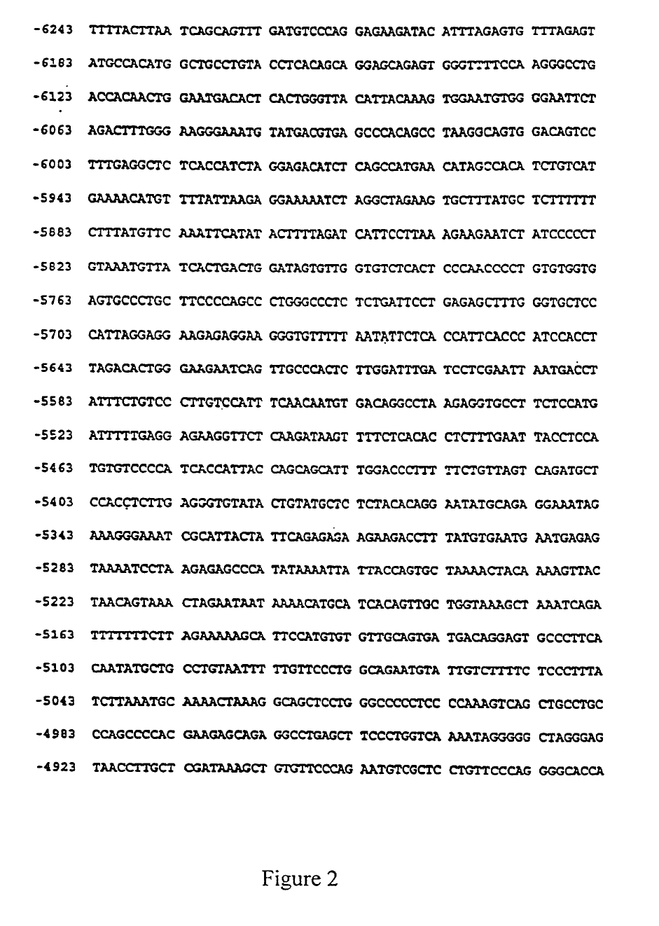

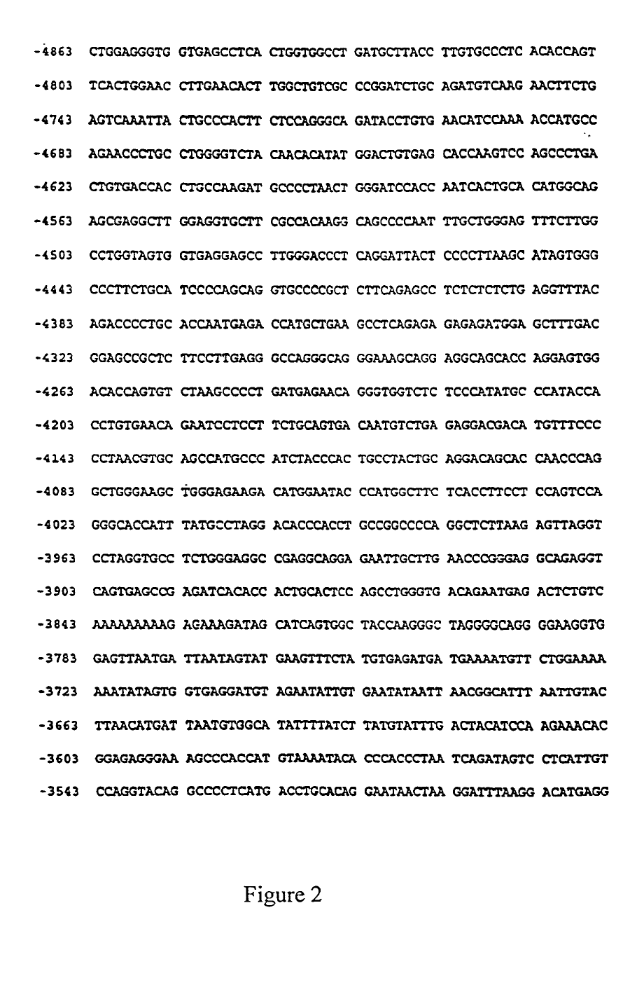

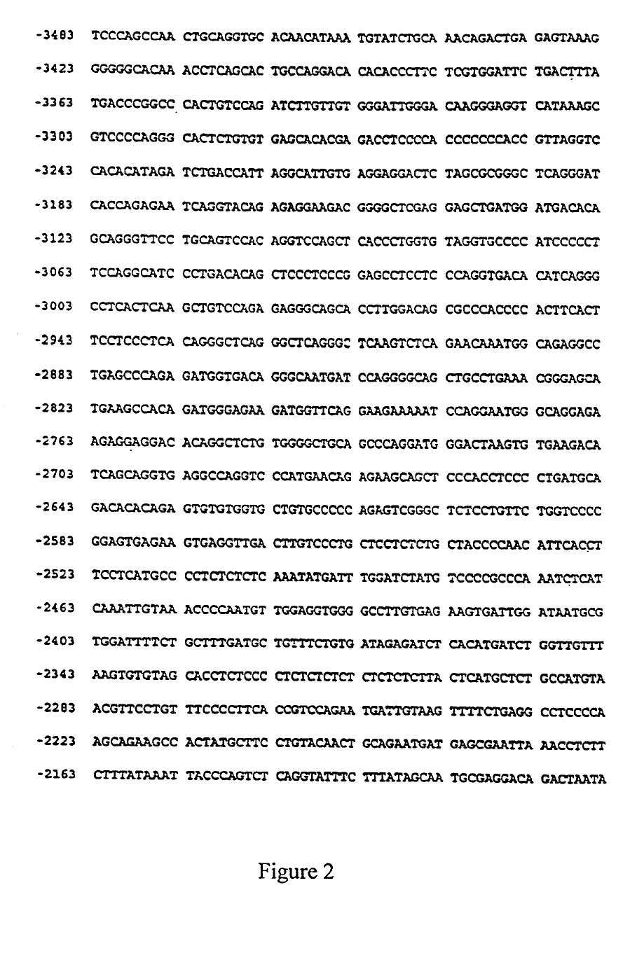

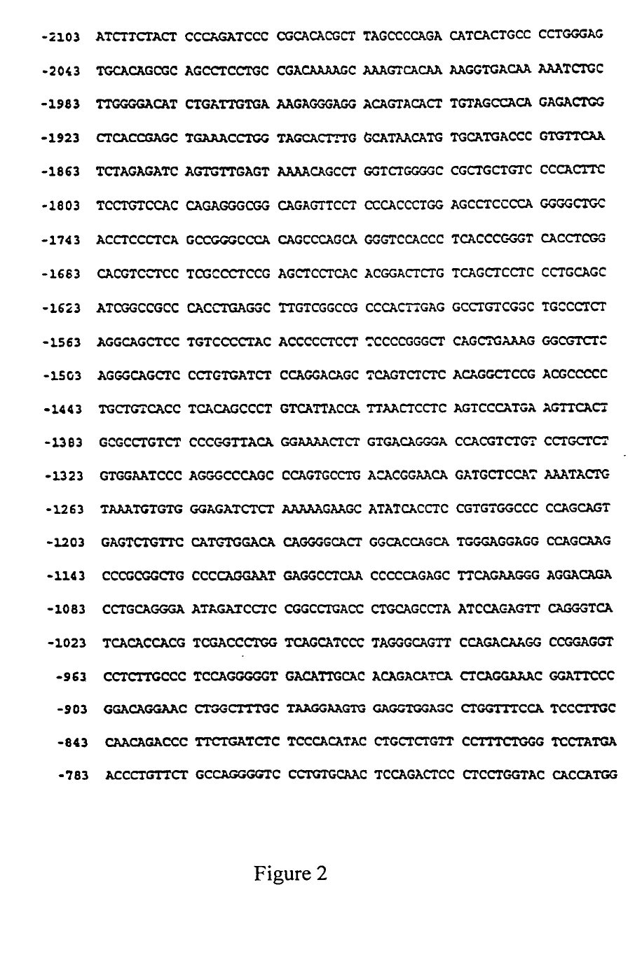

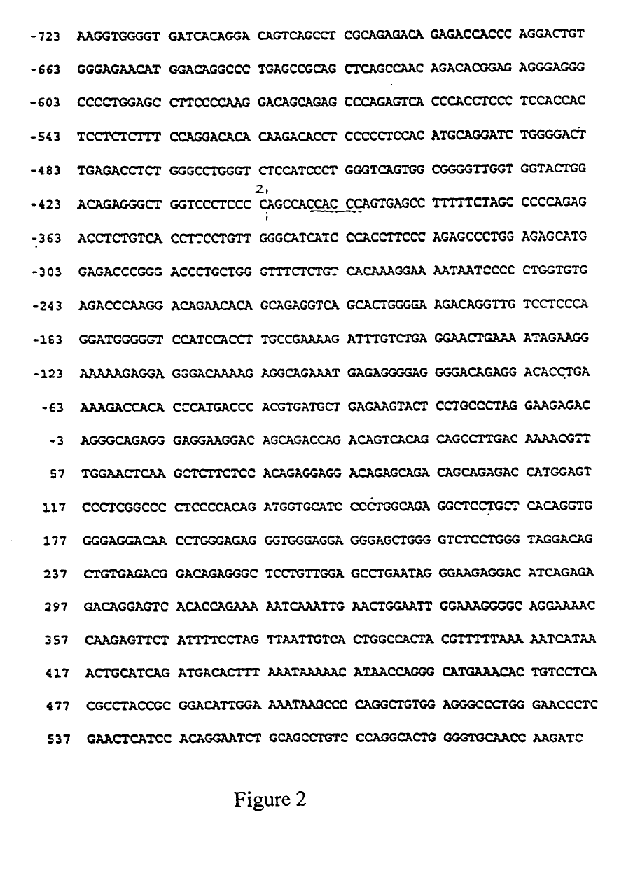

The CEA-TREs used in this invention are derived from mammalian cells, including, but not limited to, human, rat and mouse. Preferably, the CEA-TRE is human. The cloning and characterization of CEA sequences have been described in the literature and are thus made available for practice of this invention and need not be described in detail herein. The entire 5° CEA flanking region (containing the promoter, putative silencer, and enhancer elements) appears to be contained within approximately 14.5 kb upstream from the transcription start site, and is depicted in FIG. 2. Richards et al. (1995); WO 95/14100. Schrewe et al. (1990) demonstrated that a 424-bp fragment (first 424 nucleotides upstream of the translational start site) in the 5′ flanking region of the gene had higher promoter activity in CEA-producing cells than in non-producing HeLa cells. Further characterization of the 5′ flanking region of the CEA gene by Richards et al. (1995) indicated two upstream regions, about −13.6 to about −10.7 kb or about −6.1 to about −4.0 kb, when operably linked to the multimerized promoter resulted in high-level and selective expression of a reporter construct in CEA-producing LoVo and SW1463 cells. Richards et al. (1995) also localized the promoter to about −90 and about +69 relative to the transcriptional start site, with region about −41 to about −18 as essential for expression. WO95/14100 describes a series of 5′ flanking CEA fragments which confer cell-specific activity, which are herein defined as enhancers or CEA-TRE enhancers, such as about −299 to about +69; about −90 to about +69; about −14.5 to about −10.6; about −13.6 to about −10.6, about −6.1 to about −3.8. The following combinations of regions are examples of CEA-TREs (numbering according to FIG. 2): (a) about −299 to about +69; (b) about −1664 to about +69; (c) about −14462 to about −10691 plus about −299 to about +69; (d) about −89 to about −40 plus about −90 to about +69; (e) [3×(about −89 to about −40)] plus about −90 to about +69; (f) about −3919 to about −6071 plus about −299 to about +69; (g) about −6071 to about −3919 plus about −299 to about +69; (h) about −13579 to about −10691 plus about −89 to about −40 plus about −90 to about +69; (i) about −10691 to about −13579 plus about −89 to about −40 plus about −90 to about +69; (j) about −14500 to about −10600 plus about −6100 to about −3900 plus about −299 to about +69; (k) about −13600 to about −10600 plus about −6100 to about −3900 plus about −299 to about +69; (l) about −3900 to about −6100 plus [4×(about −90 to about +69)]; (m) about −13600 to about −10600 plus [4×(about −90 to about +69)]. Thus, any of the above CEA-TREs may be used in the invention as long as requisite desired functionality is displayed in the adenoviral vector.

In one embodiment, the CEA-TRE comprises an approximately 0.5 kb promoter (about −402 to about +69, SEQ ID NO: 1), which is specific for CEA-producing cells. Accordingly, the invention also includes an adenovirus vector in which the CEA-TRE comprises SEQ ID NO: 1.

A CEA-TRE can also comprise multimers. For example, a CEA-TRE can comprise a tandem series of at least two, at least three, at least four, or at least five CEA promoter fragments (such as the 440-bp fragment described by Schrewe et al.). Alternatively, a CEA-TRE could have one or more CEA promoters along with one or more CEA enhancers.

A CEA-TRE of the present invention may or may not lack a silencer. The presence of a silencer (i.e., a negative regulatory element) can assist in shutting off transcription (and thus replication) in non-permissive (i.e., non-CEA-producing) cells. Thus, presence of a silencer can confer enhanced cell-specific replication by more effectively preventing adenoviral vector replication in non-target cells. Alternatively, lack of a silencer may assist in effecting replication in target cells, thus conferring enhanced cell-specific replication due to more effective replication in target cells. Hauck et al. (1995) have described a region between about −1098 and about −403 that repressed all activity. J. Biol. Chem. 270:3602-3610.

As is readily appreciated by one skilled in the art, the CEA-TRE is a polynucleotide sequence, and, as such, can exhibit function over a variety of sequence permutations. Methods of nucleotide substitution, addition, and deletion are known in the art, and readily available functional assays (such as the CAT or luciferase reporter gene assay) allow one of ordinary skill to determine whether a sequence variant exhibits requisite cell-specific transcription function. Hence, the invention also includes adenovirus vectors comprising functionally preserved variants of the CEA-TRE nucleic acid sequences disclosed herein, which include nucleic acid substitutions, additions, and/or deletions. It is possible that certain base modifications will result in enhanced expression levels or cell-specificity. Achievement of enhanced expression levels may be especially desirable in the case of more aggressive forms of CEA-associated tumors, or when a more rapid and/or aggressive pattern of cell killing is warranted (due to an immunocompromised condition of the individual, for example).

Various replication-competent adenovirus vectors can be made according to the present invention in which a single or multiple adenovirus gene(s) are under control of a CEA-TRE.

For example, a CEA-TRE can be introduced into an adenovirus vector immediately upstream of and operably linked to a gene which is a replication gene, e.g. an early gene such as E1A or E1B or a late gene such as L1, L2, L3, L4, or L5. Optionally, the endogenous adenovirus promoter for the replication gene is deleted, placing the gene under sole transcriptional control of a CEA-TRE. Alternatively, a CEA-TRE can be placed immediately upstream of and operably linked to an ADP (adenovirus death protein) gene.

Various other replication-competent adenovirus vectors can be made according to the present invention in which, in addition to having an adenovirus gene under control of a CEA-TRE, at least one additional gene is placed under control of at least one additional heterologous (non-adenovirus) TRE. This additional TRE(s) can be a cell-, tissue-, and/or cancer-specific TRE. This additional TRE(s) can be another CEA-TRE. Optionally, the additional CEA-TRE(s) differ from the first. In this way, for example, the possibility of homologous recombination with concomitant loss of intervening sequences can be avoided. The first and additional CEA-TREs can, for example, differ in sequence in essential or non-essential regions. For example, the first CEA-TRE could comprise a CEA enhancer and a non-CEA promoter; an additional CEA-TRE could comprise a non-CEA enhancer and a CEA promoter. Alternatively, the essential portions of the promoter and/or enhancer could be identical in both, with the intervening non-essential regions different. In one embodiment, where one CEA-TRE mediates transcription of one gene, and at least one other CEA-TRE mediates transcription of another gene, the orientation of the genes is divergent or convergent, rather than tandem. In this way, any recombination between the CEA-TREs is unlikely to result to deletion of the intervening sequences.

In some embodiments, the invention provides adenoviral vectors which comprise an additional adenovirus gene under transcriptional control of a second CEA-TRE. Examples of an additional adenovirus gene under transcription control is ADP (discussed above) and genes necessary for replication, such as early genes. For example, an adenoviral vector can be constructed such that a first CEA-TRE regulates transcription of one early gene, such as E1A or E1B, and a second CEA-TRE regulates transcription of another early gene. These multiple constructs may be more desirable in that they provide more than one source of cell specificity with respect to replication.

For example, a CEA-TRE can be introduced into an adenovirus vector immediately upstream of and operably linked to an early gene such as E1A, and at least one other CEA-TRE with a different sequence can be introduced immediately upstream of and operably linked to another early gene such as E1B.

Various other replication-competent adenovirus vectors can be made according to the present invention in which, in addition to having a single or multiple adenovirus gene(s) under control of a CEA-TRE, a reporter gene(s) are also under control of a CEA-TRE.

For example, a CEA-TRE can be introduced into an adenovirus vector immediately upstream of and operably linked to an early gene such as E1A or E1B, and this construct may also contain at least one other CEA-TRE driving expression of a reporter gene. The reporter gene can encode a reporter protein, including, but not limited to, chloramphenicol acetyl transferase (CAT), β-galactosidase (encoded by the lacZ gene), luciferase, alkaline phosphatase, green fluorescent protein, and horse radish peroxidase. For detection of a putative cancer cell(s) in a biological sample, the biological sample may be treated with modified adenoviruses in which a reporter gene (e.g., luciferase) is under control of a CEA-TRE. The CEA-TRE will be transcriptionally active in cells that allow a CEA-TRE to function, and luciferase will be produced. This production will allow detection of cells producing CEA, such as tumor cells, in, for example, a human host or a biological sample. Alternatively, an adenovirus can be constructed in which a gene encoding a product conditionally required for survival (e.g., an antibiotic resistance marker) is under transcriptional control of a CEA-TRE. When this adenovirus is introduced into a biological sample, cells producing CEA will become antibiotic resistant. An antibiotic can then be introduced into the medium to kill non-CEA-producing (i.e. non-cancerous) cells.

As an example of how CEA-TRE activity can be determined, a polynucleotide sequence or set of such sequences can be generated using methods known in the art, such as chemical synthesis, site-directed mutagenesis, PCR, and/or recombinant methods. The sequence(s) to be tested is inserted into a vector containing an appropriate reporter gene, including, but not limited to, chloramphenicol acetyl transferase (CAT), β-galactosidase (encoded by the lacZ gene), luciferase (encoded by the luc gene), green fluorescent protein, alkaline phosphatase, and horse radish peroxidase. Such vectors and assays are readily available, from, inter alia, commercial sources. Plasmids thus constructed are transfected into a suitable host cell to test for expression of the reporter gene as controlled by the putative CEA-TRE using transfection methods known in the art, such as calcium phosphate precipitation, electroporation, liposomes (lipofection) and DEAE dextran. Suitable host cells include any cell type that produces CEA, including but not limited to, SW403, SW1463, SW837, NCIH508, LoVo, MKN1, MKN28, MKN45, and A549. Non-CEA-producing cells, such as LNCaP, HBL-100, HLF, HLE, 3T3, Hep3B, HuH7, CADO-LC9, and HeLa are used as a control. Results are obtained by measuring the level of expression of the reporter gene using standard assays. Comparison of expression between CEA-producing cells and control indicates presence or absence of transcriptional activation.

By “transcriptional activation” or “increase in transcription,” it is intended that transcription is increased above basal levels in the target cell (i.e., CEA-producing cell) by at least about 2-fold, preferably at least about 5-fold, preferably at least about 10-fold, more preferably at least about 20-fold, more preferably at least about 50-fold, more preferably at least about 100-fold, more preferably at least about 200-fold, even more preferably at least about 400-to about 500-fold, even more preferably at least about 1000-fold. Comparisons between or among various CEA-TREs can be assessed by measuring and comparing levels of expression within a single CEA-producing cell line. It is understood that absolute transcriptional activity of a CEA-TRE will depend on several factors, such as the nature of the target cell, delivery mode and form of the CEA-TRE, and the coding sequence that is to be selectively transcriptionally activated. To compensate for various plasmid sizes used, activities can be expressed as relative activity per mole of transfected plasmid. Alternatively, the level of transcription (i.e., mRNA) can be measured using standard Northern analysis and hybridization techniques. Levels of transfection (i.e., transfection efficiencies) are measured by co-transfecting a plasmid encoding a different reporter gene under control of a different TRE, such as the CMV immediate early promoter. This analysis can also indicate negative regulatory regions, i.e., silencers.

Alternatively a putative CEA-TRE can be assessed for its ability to confer adenoviral replication preference for cells that allow a CEA-TRE to function. For this assay, constructs containing an adenovirus gene essential to replication operatively linked to a putative CEA-TRE are transfected into cells that express CEA. Viral replication in those cells is compared, for example, to viral replication by wild type adenovirus in those cells and/or viral replication by the construct in cells not expressing CEA. A more detailed description of this kind of assay is in Example 2.

It is understood that, to practice this invention, it is not necessary to use CEA-TREs having maximum activity, or having minimum size. The requisite degree of activity is determined, inter alia, by the anticipated use and desired result. For example, if an adenoviral vector of the invention is used to monitor cells for CEA-producing activity, it is possible that less than a maximal degree of responsiveness by a CEA-TRE will suffice to qualitatively indicate the presence of such cells. Similarly, if used for treatment or palliation of a disease state, less-than-maximal responsiveness may be sufficient for the desired result, if, for example, the CEA-producing cells are not especially virulent and/or the extent of disease is relatively confined.

The size of CEA-TREs will be determined in part by the capacity of the adenoviral vector, which in turn depends upon the contemplated form of the vector (see below). Generally a minimal size is preferred, as this provides potential room for insertion of other sequences which may be desirable, such as transgenes (discussed below) or additional regulatory sequences. However, if no additional sequences are contemplated, or if, for example, an adenoviral vector will be maintained and delivered free of any viral packaging constraints, a larger CEA-TRE may be used as long as the resultant adenoviral vector is rendered replication-competent.

If no adenovirus sequences have been deleted, an adenoviral vector can be packaged with extra sequences totaling up to about 5% of the genome size, or approximately 1.8 kb. If non-essential sequences are removed from the adenovirus genome, then an additional 4.6 kb of insert can be tolerated (i.e., a total of about 1.8 kb plus 4.6 kb, which is about 6.4 kb). Examples of non-essential adenoviral sequences that can be deleted are E3 and E4 (as long as the E4 ORF6 is maintained). Preferably, a CEA-TRE will comprise a polynucleotide sequence of about 2.5 kb, more preferably about 1 kb, more preferably about 0.8 kb, even more preferably about 0.5 kb or even smaller, as a region of only about 160 bp (about −90 to about +69 of FIG. 2) has been shown to be functional.

In order to minimize non-specific replication, endogenous (i.e., adenovirus) TREs should preferably be removed. This would also provide more room for inserts in an adenoviral vector, which may be of special concern if an adenoviral vector will be packaged as a virus (see below). Even more importantly, deletion of endogenous TREs would prevent a possibility of a recombination event whereby a CEA-TRE is deleted and the endogenous TRE assumes transcriptional control of its respective adenovirus coding sequences (thus allowing non-specific replication). In one embodiment, an adenoviral vector of the invention is constructed such that the endogenous transcription control sequences of an adenoviral gene(s) are deleted and replaced by a CEA-TRE. However, endogenous TREs may also be maintained in the adenovirus vector(s), provided that sufficient cell-specific replication preference is preserved. These embodiments can be constructed by providing a CEA-TRE in addition to the endogenous TREs, preferably with the CEA-TRE intervening between the endogenous TREs and the replication gene coding segment. Requisite cell-specific replication preference is indicated by conducting assays that compare replication of the adenovirus vector in a cell that allow a CEA-TRE to function with replication in a non-CEA-producing cell. Generally, a replication differential of at least 2-fold is preferred; more preferably, at least 5-fold; more preferably, at least 10-fold; more preferably, at least 50-fold; even more preferably, at least 100-fold; still more preferably, at least 200-fold; still more preferably, at least about 400-fold to about 500-fold; even more preferably, at least 1000-fold. The acceptable differential can be determined empirically (using, for example, Northern assays or other assays known in the art or assays described in the Example section) and will depend upon the anticipated use of the adenoviral vector and/or the desired result.

Suitable target cells are any cell type that allows a CEA-TRE to function, such as cells that express, or produce, or are capable of expressing or producing CEA. Especially preferred are CEA-associated tumor (carcinoma) cells including, but not limited to, colorectal, gastric, pancreatic, lung, and breast cells and any metastases of the foregoing. CEA production can be measured using assays standard in the art, such as RIA, ELISA or Western blots (immunoassays) to determine levels of CEA protein production or Northern blots to determine levels of CEA mRNA production. Alternatively, such cells can be identified and/or characterized by their ability to transcriptionally activate a CEA-TRE (i.e., allow a CEA-TRE to function).

Any of the various serotypes of adenovirus can be used, such as Ad2, Ad5, Ad12, and Ad40. For purposes of illustration, the serotype Adenovirus 5 (Ad5) is exemplified herein.

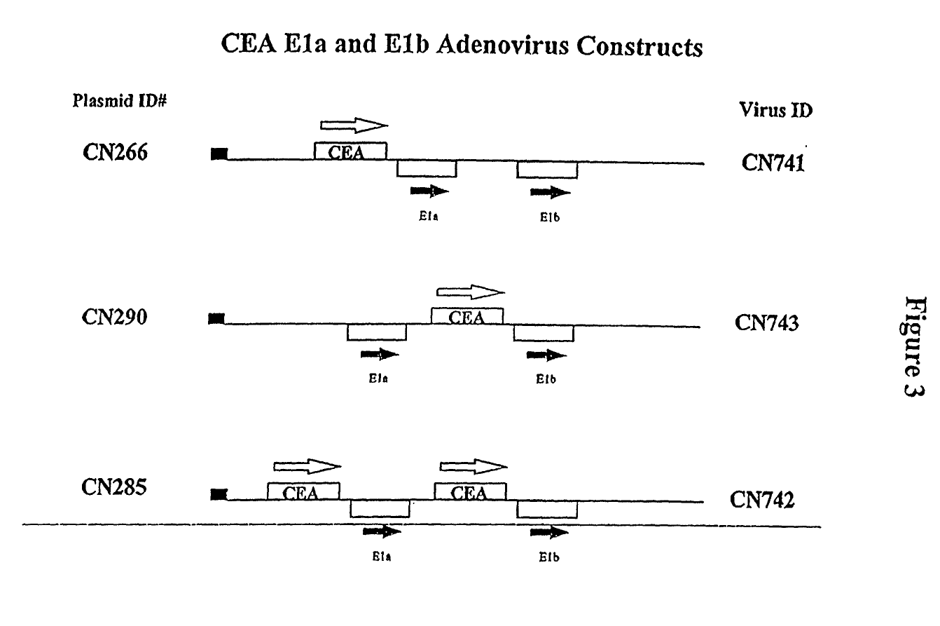

In some embodiments, a CEA-TRE is used with an adenovirus gene that is essential for propagation, so that replication competence is preferentially achievable in a target cell that allow a CEA-TRE to function. Preferably, the gene is an early gene, such as E1A, E1B, E2, or E4. (E3 is not essential for viral replication.) More preferably, the early gene under CEA-TRE control is E1A and/or E1B. More than one early gene can be placed under control of a CEA-TRE. Example 1 (FIG. 3) provides a more detailed description of such constructs.

The E1A gene is expressed immediately after viral infection (0-2 hours) and before any other viral genes. E1A protein acts as a trans-acting positive-acting transcriptional regulatory factor, and is required for the expression of the other early viral genes E1B, E2, E3, E4, and the promoter-proximal major late genes. Despite the nomenclature, the promoter proximal genes driven by the major late promoter are expressed during early times after Ad5 infection. Flint (1982) Biochem. Biophys. Acta 651:175-208; Flint (1986) Advances Virus Research 31:169-228; Grand (1987) Biochem. J. 241:25-38. In the absence of a functional E1A gene, viral infection does not proceed, because the gene products necessary for viral DNA replication are not produced. Nevins (1989) Adv. Virus Res. 31:35-81. The transcription start site of Ad5 E1A is at 498 and the ATG start site of the E1A protein is at nt 560 in the virus genome.

The E1B protein functions in trans and is necessary for transport of late mRNA from the nucleus to the cytoplasm. Defects in E1B expression result in poor expression of late viral proteins and an inability to shut off host cell protein synthesis. The promoter of E1B has been implicated as the defining element of difference in the host range of Ad40 and Ad5: clinically Ad40 is an enterovirus, whereas Ad5 causes acute conjunctivitis. Bailey, Mackay et al. (1993) Virology 193:631; Bailey et al. (1994) Virology 202:695-706. E1B proteins are also necessary to overcome restrictions imposed on viral replication by the host cell cycle and also to reduce the apoptotic effects of E1A. Goodrum et al. (1997) J. Virology 71:548-561. The E1B promoter of Ad5 consists of a single high-affinity recognition site for Spl and a TATA box.

The E2 region of adenovirus codes for proteins related to replication of the adenoviral genome, including the 72-kDa DNA-binding protein, the 80-kDa precursor terminal protein and the viral DNA polymerase. The E2 region of Ad5 is transcribed in a rightward orientation from two promoters, termed E2 early and E2 late, mapping at 76.0 and 72.0 map units, respectively. While the E2 late promoter is transiently active during late stages of infection and is independent of the E1A transactivator protein, the E2 early promoter is crucial during the early phases of viral replication.

The E2 early promoter, mapping in Ad5 from 27050-27150, consists of a major and a minor transcription initiation site, the latter accounting for about 5% of the E2 transcripts, two non-canonical TATA boxes, two E2F transcription factor binding sites and an ATF transcription factor binding site.

For a detailed review of the E2 promoter architecture see Swaminathan et al., Curr. Topics in Micro. and Imm. (1995) 199 part 3:177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kDa protein on the counterstrand. Notably, the SpeI restriction site (Ad5 position 27082) is part of the stop codon for the above mentioned 33 kDa protein and conveniently separates the major E2 early transcription initiation site and TATA-binding protein site from the upstream transcription factor binding sites E2F and ATF. Therefore, insertion of a CEA-TRE having SpeI ends into the SpeI site in the 1-strand would disrupt the endogenous E2 early promoter of Ad5 and should allow AR-restricted expression of E2 transcripts.

The E4 gene produces a number of transcription products. The E4 region codes for two polypeptides which are responsible for stimulating the replication of viral genomic DNA and for stimulating late gene expression. The protein products of open reading frames (ORFs) 3 and 6 can both perform these functions by binding the 55-kDa protein from E1B and heterodimers of E2F-1 and DP-1. The ORF 6 protein requires interaction with the E1B 55-kDa protein for activity while the ORF 3 protein does not. In the absence of functional protein from ORF 3 and ORF 6, plaques are produced with an efficiency less than 10⁻⁶ that of wild type virus. To further restrict viral replication to CEA-producing cells, E4 ORFs 1-3 can be deleted, making viral DNA replication and late gene synthesis dependent on E4 ORF 6 protein. By combining such a vector with sequences in which the E1B region is regulated by a CEA-TRE, a virus can be obtained in which both the E1B function and E4 function are dependent on an CEA-TRE driving E1B.

The major late genes relevant to the subject invention are L1, L2, L3, L4 and L5 which encode proteins of the Ad5 virus virion. All of these genes (typically coding for structural proteins) are probably required for adenoviral replication. The late genes are all under the control of the major late promoter (MLP), which is located in Ad5 at about +5986 to about +6048.

In one embodiment, an early gene such as E1A or E1B gene is under control of a CEA-TRE.

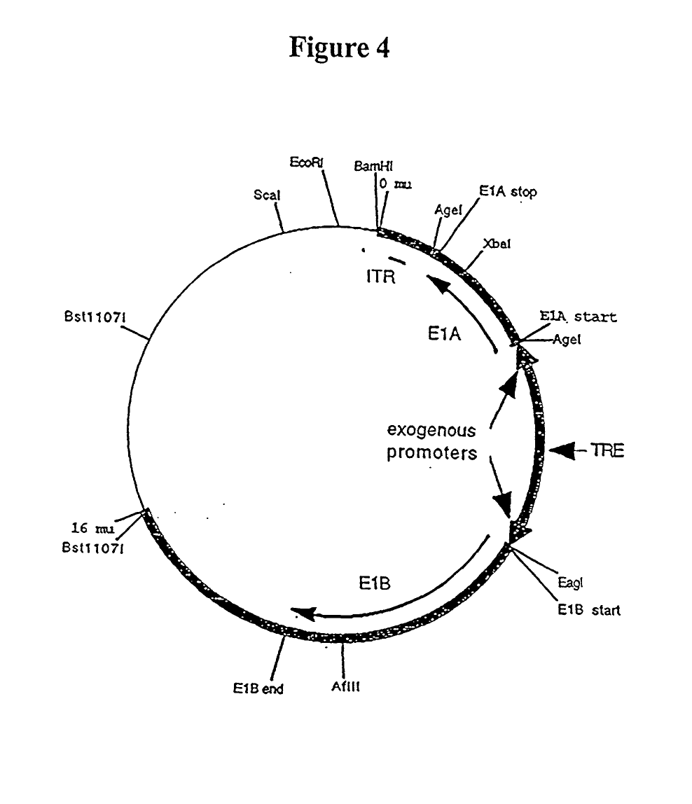

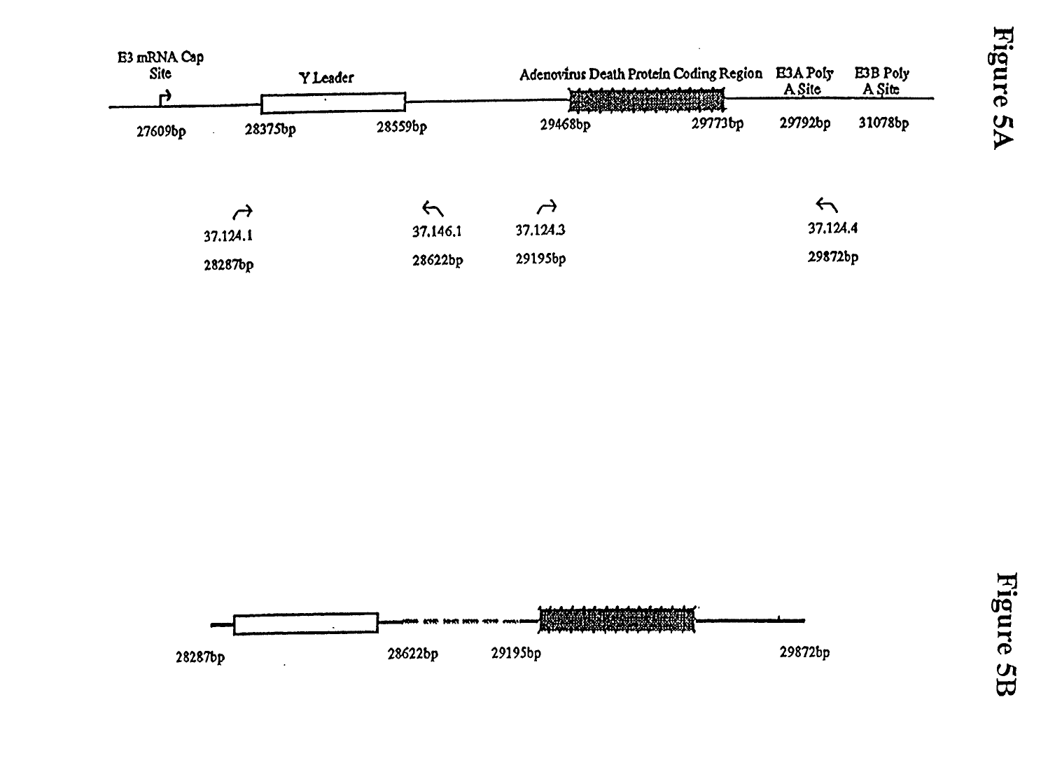

In one embodiment, E1A and E1B are under control of one or more CEA-TREs by making the following construct. A fragment containing the coding region of E1A through the E1B promoter is excised from the Ad genome and reinserted in opposite orientation (FIG. 4). In this configuration, the E1A and E1B promoters are next to each other, followed by E1A in opposite orientation (so that neither the E1A or E1B promoters are operatively linked to E1A), followed by E1B in opposite orientation with respect to E1A. An CEA-TRE(s) can be inserted between E1A and E1B coding regions, (which are in opposite orientation), so that these regions ore under control of the TRE(s). Appropriate promoter sequences are inserted proximal to the E1A and E1B region as shown in FIG. 4. Thus, an CEA-TRE may drive both E1A and E1B. Such a configuration may prevent, for example, possible loop-out events that may occur if two CEA-TREs were inserted in intact (native) Ad genome, one each 5′ of the coding regions of E1A and E1B. By introducing a polycloning site between E1A and E1B, other types of TREs can be inserted, such as a carcinogen embryonic antigen TRE (CEA-TRE); a mucin TRE (MU-TRE); or other cell-specific regulatory elements, preferably those associated with a disease state, such as neoplasm. Thus, this construct may find general use for cell-specific, temporal, or other means of control of adenovirus genes E1A and E1B, thereby providing a convenient and powerful way to render adenoviral replication dependent upon a chosen transcriptional parameter.

In addition to conferring selective cytotoxic and/or cytolytic activity by virtue of preferential replication competence in cells that allow a CEA-TRE to function, the adenovirus vectors of this invention can further include a heterologous gene (transgene) under the control of a CEA-TRE. In this way, various genetic capabilities may be introduced into target cells that allow a CEA-TRE to function, particularly CEA-associated tumor cells, such as CEA-associated carcinoma cells. For example, in certain instances, it may be desirable to enhance the degree and/or rate of cytotoxic activity, due to, for example, the relatively refractory nature or particular aggressiveness of the CEA-producing target cell. This could be accomplished by coupling the cell-specific replicative cytotoxic activity with cell-specific expression of, for example, HSV-tk and cytosine deaminase (cd), which renders cells capable of metabolizing 5-fluorocytosine (5-FC) to the chemotherapeutic agent 5-fluorouracil (5-FU). Using these types of transgenes may also confer a bystander effect.

Other desirable transgenes that may be introduced via an adenovirus vector(s) include genes encoding cytotoxic proteins, such as the A chains of diphtheria toxin, ricin or abrin [Palmiter et al. (1987) Cell 50: 435; Maxwell et al. (1987) Mol. Cell. Biol. 7: 1576; Behringer et al. (1988) Genes Dev. 2: 453; Messing et al. (1992) Neuron 8: 507; Piatak et al. (1988) J. Biol. Chem. 263: 4937; Lamb et al. (1985) Eur. J. Biochem. 148: 265; Frankel et al. (1989) Mol. Cell. Biol. 9: 415], genes encoding a factor capable of initiating apoptosis, sequences encoding antisense transcripts or ribozymes, which among other capabilities may be directed to mRNAs encoding proteins essential for proliferation, such as structural proteins, or transcription factors; viral or other pathogenic proteins, where the pathogen proliferates intracellularly, genes that encode an engineered cytoplasmic variant of a nuclease (e.g. RNase A) or protease (e.g. awsin, papain, proteinase K, carboxypeptidase, etc.), or encode the Fas gene, and the like. Other genes of interest include cytokines, antigens, transmembrane proteins, and the like, such as IL-1, -2, -6, -12, GM-CSF, G-CSF, M-CSF, IFN-α, -β, -γ, TNF-α, -β, TGF-α, -β, NGF, and the like. The positive effector genes could be used in an early phase, followed by cytotoxic activity due to replication.

In some embodiments, the adenovirus death protein (ADP), encoded within the E3 region, is maintained (i.e. contained) in the adenovirus vector. The ADP gene, under control of the major late promoter (MLP), appears to code for a protein (ADP) that is important in expediting host cell lysis. Tollefson et al. (1996) J. Virol. 70(4):2296; Tollefson et al. (1992) J. Virol. 66(6):3633. Thus, adenoviral vectors containing the ADP gene may render the adenoviral vector more potent, making possible more effective treatment and/or a lower dosage requirement.

Accordingly, the invention provides a adenoviral vectors that includes a polynucleotide sequence encoding an ADP. A DNA sequence encoding an ADP and the amino acid sequence of an ADP are depicted in SEQ ID NO: 19 and SEQ ID NO:20, respectively. Briefly, an ADP coding sequence is obtained preferably from Ad2 (since this is the strain in which ADP has been more fully characterized) using techniques known in the art, such as PCR. Preferably, the Y leader (which is an important sequence for correct expression of late genes) is also obtained and ligated to the ADP coding sequence. The ADP coding sequence (with or without the Y leader) can then be introduced into the adenoviral genome, for example, in the E3 region (where the ADP coding sequence will be driven by the MLP or the E3 promoter). The ADP coding sequence could also be inserted in other locations of the adenovirus genome, such as the E4 region. Alternatively, the ADP coding sequence could be operably linked to a heterologous promoter (with or without enhancer(s)), including, but not limited to, another viral promoter, a tissue specific promoter such as AFP (alpha fetoprotein), carcinoembryonic antigen (CEA), mucin, and rat probasin. Example 5 provides a description of an ADP construct in which the coding sequence for ADP was inserted into the E3 region of Ad5.

In some embodiments, the invention provides adenoviral vectors which comprise an additional adenovirus gene under transcriptional control of a second CEA-TRE. Examples of an additional adenvirus gene under transcriptional control is ADP (discussed above) and genes necessary for replication, such as early genes. For example, an adenoviral vector can be constructed such that a first CEA-TRE regulates transcription of one early gene, such as E1A or E1B, and a second CEA-TRE regulates transcription of another early gene. These multiple constructs may be more desirable in that they provide more than one source of cell specificity with respect to replication

The adenoviral vectors can be used in a variety of forms, including, but not limited to, naked polynucleotide (usually DNA) constructs. Adenoviral vectors can, alternatively, comprise polynucleotide constructs that are complexed with agents to facilitate entry into cells, such as cationic liposomes or other cationic compounds such as polylysine; packaged into infectious adenovirus particles (which may render the adenoviral vector(s) more immunogenic); packaged into other particulate viral forms such as HSV or AAV; complexed with agents (such as PEG) to enhance or dampen an immune response; complexed with agents that facilitate in vivo transfection, such as DOTMA™, DOTAP™, and polyamines.

Thus, the invention also provides an adenovirus capable of replicating preferentially in CEA-producing cells. “Replicating preferentially” means that the adenovirus replicates more in a CEA-producing cell than a non-CEA-producing cell. Preferably, the adenovirus replicates at a significantly higher rate in CEA-producing cells than non CEA-producing cells; preferably, at least about 2-fold higher, preferably, at least about 5-fold higher, more preferably, at least about 10-fold higher, still more preferably at least about 50-fold higher, even more preferably at least about 100-fold higher, still more preferably at least about 400- to 500-fold higher, still more preferably at least about 1000-fold higher, most preferably at least about 1×10⁶ higher. Most preferably, the adenovirus replicates solely in CEA-producing cells (that is, does not replicate or replicates at a very low levels in non CEA-producing cells).

If an adenoviral vector comprising an adenovirus polynucleotide is packaged into a whole adenovirus (including the capsid), the adenovirus itself may also be selected to further enhance targeting. For example, adenovirus fibers mediate primary contact with cellular receptor(s) aiding in tropism. See, e.g., Amberg et al. (1997) Virol. 227:239-244. If a particular subgenus of an adenovirus serotype displayed tropism for a target cell type and/or reduced affinity for non-target cell types, such subgenus(or subgenera) could be used to further increase cell-specificity of cytotoxicity and/or cytolysis.

The adenoviral vectors may be delivered to the target cell in a variety of ways, including, but not limited to, liposomes, general transfection methods that are well known in the art, such as calcium phosphate precipitation, electroporation, direct injection, and intravenous infusion. The means of delivery will depend in large part on the particular adenoviral vector (including its form) as well as the type and location of the target cells (i.e., whether the cells are in vitro or in vivo).

If used in packaged adenoviruses, adenovirus vectors may be administered in an appropriate physiologically acceptable carrier at a dose of about 10⁴ to about 10¹⁴. The multiplicity of infection will generally be in the range of about 0.001 to 100. If administered as a polynucleotide construct (i.e., not packaged as a virus) about 0.01 μg to about 1000 μg of an adenoviral vector can be administered. The adenoviral vector(s) may be administered one or more times, depending upon the intended use and the immune response potential of the host or may be administered as multiple, simultaneous injections. If an immune response is undesirable, the immune response may be diminished by employing a variety of immunosuppressants, so as to permit repetitive administration, without a strong immune response. If packaged as another viral form, such as HSV, an amount to be administered is based on standard knowledge about that particular virus (which is readily obtainable from, for example, published literature) and can be determined empirically.

The present invention also provides host cells comprising (i.e., transformed with) the adenoviral vectors described herein. Both prokaryotic and eukaryotic host cells can be used as long as sequences requisite for maintenance in that host, such as appropriate replication origin(s). are present. For convenience, selectable markers are also provided. Host systems are known in the art and need not be described in detail herein. Prokaryotic host cells include bacterial cells, for example, E. coli, B. subtilis, and mycobacteria. Among eukaryotic host cells are yeast, insect, avian, plant, C. elegans (or nematode) and mammalian host cells. Examples of fungi (including yeast) host cells are S. cerevisiae, Kluyveromyces lactis (K. lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, mouse L cells, LNCaP cells, Chinese hamster ovary (CHO) cells, human embryonic kidney (HEK) cells, and African green monkey cells. Xenopus laevis oocytes, or other cells of amphibian origin, may also be used. Suitable host cells also include any cells that allow a CEA-TRE to function such as those that produce CEA or any protein that is known to activate a CEA-TRE (whether this protein is produced naturally or recombinantly).

The present invention also includes compositions, including pharmaceutical compositions, containing the adenoviral vectors described herein. Such compositions are useful for administration in vivo, for example, when measuring the degree of transduction and/or effectiveness of cell killing in an individual. Preferably, these compositions further comprise a pharmaceutically acceptable excipient. These compositions, which can comprise an effective amount of an adenoviral vector of this invention in a pharmaceutically acceptable excipient, are suitable for systemic administration to individuals in unit dosage forms, sterile parenteral solutions or suspensions, sterile non-parenteral solutions or oral solutions or suspensions, oil in water or water in oil emulsions and the like. Formulations for parenteral and nonparenteral drug delivery are known in the art and are set forth in Remington's Pharmaceutical Sciences, 18th Edition, Mack Publishing (1990). Compositions also include lyophilized and/or reconstituted forms of the adenoviral vectors (including those packaged as a virus, such as adenovirus) of the invention.

The present invention also encompasses kits containing an adenoviral vector(s) of this invention. These kits can be used for diagnostic and/or monitoring purposes, preferably monitoring. Procedures using these kits can be performed by clinical laboratories, experimental laboratories, medical practitioners, or private individuals. Kits embodied by this invention allow someone to detect the presence of CEA-producing cells in a suitable biological sample, such as biopsy specimens.

The kits of the invention comprise an adenoviral vector described herein in suitable packaging. The kit may optionally provide additional components that are useful in the procedure, including, but not limited to, buffers, developing reagents, labels, reacting surfaces, means for detection, control samples, instructions, and interpretive information.

Preparation of the adenovirus vectors of the invention

The adenovirus vectors of this invention can be prepared using recombinant techniques that are standard in the art. Generally, a CEA-TRE is inserted 5′ to the adenoviral gene of interest, preferably an adenoviral replication gene, more preferably one or more early replication genes (although late gene(s) can be used). A CEA-TRE can be prepared using oligonucleotide synthesis (if the sequence is known) or recombinant methods (such as PCR and/or restriction enzymes). Convenient restriction sites, either in the natural adeno-DNA sequence or introduced by methods such as PCR or site-directed mutagenesis, provide an insertion site for a CEA-TRE. Accordingly, convenient restriction sites for annealing (i.e., inserting) a CEA-TRE can be engineered onto the 5′ and 3′ ends of a CEA-TRE using standard recombinant methods, such as PCR.

Polynucleotides used for making adenoviral vectors of this invention may be obtained using standard methods in the art, such as chemical synthesis, recombinant methods and/or obtained from biological sources.

Adenoviral vectors containing all replication-essential elements, with the desired elements (e.g., E1A) under control of a CEA-TRE, are conveniently prepared by homologous recombination or in vitro ligation of two plasmids, one providing the left-hand portion of adenovirus and the other plasmid providing the right-hand region, one or more of which contains at least one adenovirus gene under control of a CEA-TRE. If homologous recombination is used, the two plasmids should share at least about 500 bp of sequence overlap. Each plasmid, as desired, may be independently manipulated, followed by cotransfection in a competent host, providing complementing genes as appropriate, or the appropriate transcription factors for initiation of transcription from a CEA-TRE for propagation of the adenovirus. Plasmids are generally introduced into a suitable host cell such as 293 cells using appropriate means of transduction, such as cationic liposomes. Alternatively, in vitro ligation of the right and left-hand portions of the adenovirus genome can also be used to construct recombinant adenovirus derivative containing all the replication-essential portions of adenovirus genome. Berkner et al. (1983) Nucleic Acid Research 11: 6003-6020; Bridge et al. (1989) J. Virol. 63: 631-638.

For convenience, plasmids are available that provide the necessary portions of adenovirus. Plasmid pXC.1 (McKinnon (1982) Gene 19:33-42) contains the wild-type left-hand end of Ad5. PBHG10 (Bett et al. (1994); Microbix Biosystems Inc., Toronto) provides the right-hand end of Ad5, with a deletion in E3. The deletion in E3 provides room in the virus to insert a 3 kb CEA-TRE without deleting the endogenous enhancer/promoter. The gene for E3 is located on the opposite strand from E4 (r-strand). pBHG11 provides an even larger E3 deletion (an additional 0.3 kb is deleted). Bett et al. (1994).

For manipulation of the early genes, the transcription start site of Ad5 E1A is at 498 and the ATG start site of the E1A coding segment is at 560 in the virus genome. This region can be used for insertion of a CEA-TRE. A restriction site may be introduced by employing polymerase chain reaction (PCR), where the primer that is employed may be limited to the Ad5 genome, or may involve a portion of the plasmid carrying the Ad5 genomic DNA. For example, where pBR322 is used, the primers may use the EcoRI site in the pBR322 backbone and the XbaI site at nt 1339 of Ad5. By carrying out the PCR in two steps, where overlapping primers at the center of the region introduce a 30 sequence change resulting in a unique restriction site, one can provide for insertion of CEA-TRE at that site. Example 1 provides a more detailed description of an adenoviral vector in which E1A is under CEA-TRE control.

A similar strategy may also be used for insertion of a CEA-TRE element to regulate E1B. The E1B promoter of Ad5 consists of a single high-affinity recognition site for Spl and a TATA box. This region extends from Ad5 nt 1636 to 1701. By insertion of a CEA-TRE in this region, one can provide for cell-specific transcription of the E1B gene. By employing the left-hand region modified with the cell-specific response element regulating E1A, as the template for introducing a CEA-TRE to regulate E1B, the resulting adenovirus vector will be dependent upon the cell-specific transcription factors for expression of both E1A and E1B. Example 1 provides a more detailed description of how such constructs can be prepared.

Similarly, a CEA-TRE can be inserted upstream of the E2 gene to make its expression cell-specific. The E2 early promoter, mapping in Ad5 from 27050-27150, consists of a major and a minor transcription initiation site, the latter accounting for about 5% of the E2 transcripts, two non-canonical TATA boxes, two E2F transcription factor binding sites and an ATF transcription factor binding site (for a detailed review of the E2 promoter architecture see Swarrinathan et al., Curr. Topics in Micro. and Imm. (1995) 199(part 3):177-194.

The E2 late promoter overlaps with the coding sequences of a gene encoded by the counterstrand and is therefore not amenable for genetic manipulation. However, the E2 early promoter overlaps only for a few base pairs with sequences coding for a 33 kD protein on the counterstrand. Notably, the SpeI restriction site (Ad5 position 27082) is part of the stop codon for the above mentioned 33 kD protein and conveniently separates the major E2 early transcription initiation site and TATA-binding protein site from the upstream transcription factor binding sites E2F and ATF. Therefore, insertion of a CEA-TRE having SpeI ends into the SpeI site in the 1-strand would disrupt the endogenous E2 early promoter of Ad5 and should allow CEA-restricted expression of E2 transcripts.

For E4, one must use the right hand portion of the adenovirus genome. The E4 transcription start site is predominantly at about nt 35605, the TATA box at about nt 35631 and the first AUG/CUG of ORFI is at about nt 35532. Virtanen et al. (1984) J. Virol. 51: 822-831. Using any of the above strategies for the other genes, a CEA-TRE may be introduced upstream from the transcription start site. For the construction of full-length adenovirus with a CEA-TRE inserted in the E4 region, the co-transfection and homologous recombination are performed in W162 cells (Weinberg et al. (1983) Proc. Natl. Acad Sci. 80:5383-5386) which provide E4 proteins in trans to complement defects in synthesis of these proteins.

Methods of packaging adenovirus polynucleotides into adenovirus particles are known in the art and are described in the Examples.

Methods using the adenovirus vectors of the invention

The subject vectors can be used for a wide variety of purposes, which will vary with the desired or intended result. Accordingly, the present invention includes methods using the adenoviral vectors described above.

In one embodiment, methods are provided for conferring selective cytotoxicity in cells that allow a CEA-TRE to function, preferably cells expressing CEA, comprising contacting such cells with an adenovirus vector described herein. Cytotoxicity can be measured using standard assays in the art, such as dye exclusion, ³H-thymidine incorporation, and/or lysis.

In another embodiment, methods are provided for propagating an adenovirus specific for cells which allow an CEA-TRE to function, preferably mammalian cells expressing CEA. These methods entail combining an adenovirus vector with the cells, whereby said adenovirus is propagated.

Another embodiment provides methods for killing cells that allow a CEA-TRE to function in a mixture of cells, comprising combining the mixture of cells with an adenovirus vector of the present invention. The mixture of cells is generally a mixture of normal cells and cancerous cells that allow a CEA-TRE to function, and can be an in vivo mixture or in vitro mixture.

The invention also includes methods for detecting cells which allow a CEA-TRE to function, such as those capable of expressing CEA in a biological sample. These methods are particularly useful for monitoring the clinical and/or physiological condition of an individual (i.e., mammal), whether in an experimental or clinical setting. In one method, cells of a biological sample are contacted with an adenovirus vector, and replication of the adenoviral vector is detected. Alternatively, the sample can be contacted with an adenovirus in which a reporter gene is under control of a CEA-TRE. When such an adenovirus is introduced into a biological sample, expression of the reporter gene indicates the presence of cells that allow a CEA-TRE to function. Alternatively, an adenovirus can be constructed in which a gene conditionally required for cell survival is placed under control of a CEA-TRE. This gene may encode, for example, antibiotic resistance. Later the biological sample is treated with an antibiotic. The presence of surviving cells expressing antibiotic resistance indicates the presence of cells capable of CEA-TRE function. A suitable biological sample is one in which cells that allow a CEA-TRE to function, such as CEA-producing cells, may be or are suspected to be present. Generally, in mammals, a suitable clinical sample is one in which cancerous cells that a allow a CEA-TRE to function, such as colorectal carcinoma cells, are suspected to be present. Such cells can be obtained, for example, by needle biopsy or other surgical procedure. Cells to be contacted may be treated to promote assay conditions, such as selective enrichment, and/or solubilization. In these methods, cells that allow a CEA-TRE to function can be detected using in vitro assays that detect adenoviral proliferation, which are standard in the art. Examples of such standard assays include, but are not limited to, burst assays (which measure virus yield) and plaque assays (which measure infectious particles per cell). Propagation can also be detected by measuring specific adenoviral DNA replication, which are also standard assays.

The invention also provides methods of modifying the genotype of a target cell, comprising contacting the target cell with an adenovirus vector described herein, wherein the adenoviral vector enters the cell.

The invention further provides methods of suppressing tumor cell growth, preferably a tumor cell that allows a CEA-TRE to function, comprising contacting a tumor cell with an adenoviral vector of the invention such that the adenoviral vector enters the tumor cell and exhibits selective cytotoxicity for the tumor cell. As used herein, “tumor cells” and “tumor” refer to cells which exhibit relatively autonomous growth, so that they exhibit an aberrant growth phenotype characterized by a significant loss of control of cell proliferation. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage. “Suppressing” tumor growth indicates a growth state that is curtailed when compared to growth without contact with, i.e., transfection by, an adenoviral vector described herein.

The invention also provides methods of lowering the levels of a tumor cell marker in an individual, comprising administering to the individual an adenoviral vector of the present invention, wherein the adenoviral vector is selectively cytotoxic toward cells that allow a CEA-TRE to function. Tumor cell markers include, but are not limited to, PSA, hK2, and carcinoembryonic antigen (CEA). Methods of measuring the levels of a tumor cell marker are known to those of ordinary skill in the art and include, but are not limited to, immunological assays, such as enzyme-linked immunosorbent assay (ELISA), using antibodies specific for the tumor cell marker. In general, a biological sample is obtained from the individual to be tested, and a suitable assay, such as an ELISA, is performed on the biological sample.

The invention also provides methods of treatment, in which an effective amount of an adenoviral vector(s) described herein is administered to an individual. Treatment using an adenoviral vector(s) is indicated in individuals with CEA-associated tumors as described above, such as hepatocellular carcinoma. Also indicated are individuals who are considered to be at risk for developing CEA-associated tumors (including single cells), such as those who have had disease which has been resected and those who have had a family history of CEA-associated tumors. Determination of suitability of administering adenoviral vector(s) of the invention will depend, inter alia, on assessable clinical parameters such as serological indications and histological examination of tissue biopsies. Generally, a pharmaceutical composition comprising an adenoviral vector(s) in a pharmaceutically acceptable excipient is administered. Pharmaceutical compositions are described above.

The amount of adenoviral vector(s)to be administered will depend on several factors, such as route of administration, the condition of the individual, the degree of aggressiveness of the disease, the particular CEA-TRE employed, and the particular vector construct (i.e., which adenovirus gene(s) is under CEA-TRE control).

If administered as a packaged adenovirus, from about 10⁴ to about 10¹⁴, preferably from about 10⁴ to about 10¹², more preferably from about 10⁴to about 10¹⁰. If administered as a polynucleotide construct (i.e., not packaged as a virus), about 0.01 μg to about 100 μg can be administered, preferably 0.1 μg to about 500 μg, more preferably about 0.5 μg to about 200 μg. More than one adenoviral vector can be administered, either simultaneously or sequentially. Administrations are typically given periodically, while monitoring any response. Administration can be given, for example, intratumorally, intravenously or intraperitoneally.

The adenoviral vectors of the invention can be used alone or in conjunction with other active agents, such as chemotherapeutics, that promote the desired objective.

The following examples are provided to illustrate but not limit the invention.

### EXAMPLES

### Example 1

### Adenovirus Vectors Containing a CEA-TRE Driving Transcription of E1A and/or E1B

1.A. The carcinoembryonic antigen transcriptional response element (CEA-TRE)

The transcriptional response element of the carcinoembryonic antigen (CEA-TRE), about −402 to about +69 bp relative to the transcriptional start (SEQ ID NO:1), was amplified by polymerase chain reaction (PCR) from human genomic DNA using primers:

| | | |
|---|---|---|
| 5′ ATT ACC GGT AGC CAC CAC CCA GTG AG 3′ | (39.174B, upper primer) | (SEQ ID NO:9) and |
| 5′ TAG ACC GGT GCT TGA GTT CCA GGA AC 3′ | (39.174D) | (SEQ ID NO:10) |

A unique restriction site AgeI was introduced by the primer pair at the ends of the PCR amplified product.

The CEA-TRE PCR fragment was ligated into pGEM-T vector (Promega) which had been linearized with EcoRV. The ligation mixture was transformed into E. coli DH5α cells. The desired clone, carrying a CEA-TRE fragment, was obtained and designated CN265.

1.B. Construction of CEA-TRE adenoviruses comprising one or two adenovirus genes under transcriptional control of CEA-TRE

Three replication-competent, CEA cell-specific adenoviruses were produced:

CN741, which contains a CEA-TRE driving the expression of the E1A gene;

CN742, which contains two CEA-TREs driving expression of the E1A and E1B genes; and

CN743, which contains a CEA-TRE driving E1B expression.

The viruses were generated by homologous recombination in 293 cells and cloned by plaque purification. The structure of the genomic DNA was analyzed by PCR and sequencing of the junctions between the inserted sequences and the Ad genomic sequences to confirm that the viruses contained the desired structures.

1.B.1. CEA-TRE-driven E1A adenovirus plasmid (CN741)

Briefly, a CEA-TRE fragment was inserted into CN124 (described below) to generate CN266, which comprises the left-hand end of adenovirus with a CEA-TRE controlling expressing of the adenovirus E1A gene. CN266 was recombined with a plasmid carrying the right-hand portion of adenovirus to generate CN741, which is a full-length adenovirus in which CEA-TRE controls expression of adenovirus gene E1A.

In more detail, the CEA-TRE sequence was excised from CN265 (described in Example 1.A.) by digestion with PinAI.

CN124 is a derivative of construct pXC.1, which contains the wild-type (wt) left-hand end of Ad5, from nt (nucleotide) 22 to 5790, including both E1A and E1B [McKinnon (1982) Gene 19:33-42]. Plasmid pXC.1 was purchased from Microbix Biosystems Inc. (Toronto). We introduced an AgeI site 12 bp 5′ to the E1A initiation codon (Ad5 nt 547) by oligo-directed mutagenesis and linked PCR. To achieve this, pXC.1 was PCR-amplified using primers:

15.133A, 5′-TCGTCTTCAAGAATTCTCA (SEQ ID NO:2), containing an EcoRI site, and

15.134B, 5′-TTTCAGTCACCGGTGTCGGA (SEQ ID NO:3), containing an extra A to introduce an AgeI site.

This created a segment from the EcoRI site in the pBR322 backbone to Ad5 nt 560.

A second segment of pXC.1 from Ad nt 541 to the XbaI site at Ad nucleotide 1339 was amplified using primers:

15.133B, 5′-GCATTCTCTAGACACAGGTG (SEQ ID NO:4) containing an XbaI site, and

15.134A, 5′-TCCGACACCGGTGACTGAAA (SEQ ID NO:5), containing an extra T to introduce an AgeI site.

These two PCR-amplified DNA segments were mixed and amplified with primers 15.133A and 15.133B to create a DNA segment from the EcoRI site to the XbaI site of pXC.1. This DNA segment encompasses the leftmost 1317 bases of Ad sequence and contains an AgeI site at Ad nt 547. This DNA segment was used to replace the corresponding segment of pXC.1 to create CN95.

An EagI site was created upstream of the E1B start site by inserting a G residue at Ad5 nt 1682 by oligonucleotide directed mutagenesis as above. To simplify insertion of a CEA-TRE in the EagI site, the endogenous EagI site in CN95 was removed by digestion with EagI, treatment with mung bean nuclease, and religation to construct CN114. The following primers were used to amplify the segment between 1682 and the KpnI site at Ad5 nt 2048:

15.133A, 5′-TCGTCTTCAAGAATTCTCA (SEQ ID NO:2), containing an EcoRI site, and

9.4, 5′-GCCCACGGCCGCATTATATAC (SEQ ID NO:6), containing an EagI site

9.3, 5′-GTATATAATGCGGCCGTGGGC (SEQ ID NO:7), containing an extra G as well as an EagI site, and

24.020, 5′-CCAGAAAATCCAGCAGGTACC (SEQ ID NO:8), containing a KpnI site.

Co-amplification of the two segments with primers 15.133A and 24.020 yielded a fragment with an EagI site at Ad5 nt 1682 which was used to replace the corresponding EcoRI/KpnI site in pXC.1 to construct CN 124.

A CEA-TRE fragment excised from CN265 (see above) by digestion with PinAI was ligated into similarly digested CN124 (which contains the left hand end of the adenovirus) to generate CN266. CN266 is a vector comprising the left-hand portion of adenovirus, in which a CEA-TRE is inserted upstream of and controls expression of E1A.

The full-length CEA-E1A virus, designated CN741, was constructed by homologous recombination of CN266 and BHG11, which contains the right hand side of Adenovirus 5. Briefly, the plasmid CN266 was digested with PvuI; BHG11, with ClaI. Equivalent amounts (5 μg) of each linearly cut plasmid were transfected into 293 cells with a 4-fold excess of cationic liposomes such as Lipofectin DOTAP/DOPE (1:1). 293 is a human embryonic kidney cell line which efficiently expresses the E1A and E1B genes of Ad5 and exhibits a high transfection efficiency with adenovirus DNA. 8 days after infection, viral plaques were observed on the cell monolayer; cells/viruses were harvested, freeze-thawed 3×, centrifuged to pellet the cellular debris, and the supernatant collected. CN741 was plaque-purified three times.

In an alternative protocol for transfection, the plasmids to be combined are co-transfected into 293 cells using a 4-fold excess of cationic liposomes such as Lipofectin (DOTMA:DOPE, Life Technologies) by combining the two plasmids, then mixing the plasmid DNA solution (10 μg of each plasmid in 200 μl of minimum essential medium without serum or other additives) with an 4-molar excess of liposomes in 200 μl of the same buffer. The DNA-lipid complexes were then placed on the cells and incubated at 37° C., 5% CO₂ for 16 hours. After incubation, the medium was changed to MEM with 10% fetal bovine serum and the cells are further incubated at 37° C., 5% CO₂, for two weeks with two changes of medium. At the end of this time the cells and medium were transferred to tubes, freeze-thawed three times, and the lysate was used to infect 293 cells at the proper dilution to detect individual viruses as plaques.

Plaques obtained were plaque-purified twice, and viruses were characterized for presence of desired sequences by PCR and occasionally by DNA sequencing. For further experimentation the viruses are prepared on a larger scale by cesium chloride gradient centrifugation.

Several clones of CN741, the full-length adenovirus in which a CEA-TRE controls E1A expression, were characterized by PCR. Southern Blot, and the plaque assay for specificity.

1. PCR: Primers were used to amplify the region of clones of CN741 starting upstream of the CEA insert in the E1A region (primer 39.141C: 5′ ATT TGT CTA GGG CCG GGA CTT 3′) and downstream at the 3′ end of the E1B region (primer 39.141H: 5′CGC GCG CAA AAC CCC TAA ATA AAG 3′) of adenovirus. The amplified fragment is 4249 bp. The following clones tested positive by PCR: 46.130.7.4., 46.130.8.3, 46.130.9.1.1, 46.130.9.2.1, 46.130.9.3.1, and 46.130.9.4.1.

2. Southern blot: Positive clones of CN741 were further characterized by Southern blot. Viral DNA of CN741 clones was digested by the following enzymes: Scal, AfIII, and AfIII/XbaI. The viral DNA was probed with a randomly primed fragment of E1A. The correct fragments were as follows: ScaI digest, 926 and 5645 bp; AfIII digest, 4011 bp; and AfIII/XbaI digest, 1817 bp. Each positive clone displayed the correct fragment pattern.

3. Plaque assay: The plaque assay is described in Example 2.

These assays confirmed the identity of CN741, the full-length adenovirus in which a CEA-TRE controls E1A expression.

1.B.2. CEA-TRE-driven E1B adenovirus plasmid (CN743)

Briefly, a CEA-TRE fragment was inserted into CN124 (described above) to generate CN290, which comprises the left-hand end of adenovirus with a CEA-TRE controlling expressing of the adenovirus E1B gene. CN290 was recombined with a plasmid carrying the right-hand portion of adenovirus to generate CN743, which is a full-length adenovirus in which CEA-TRE controls expression of adenovirus gene E1B.

In more detail, the CEA-TRE was obtained as an EagI fragment from CN284 (described below). This fragment was isolated by gel electrophoresis and inserted into CN124, similarly cut with EagI. CN124, also described above, contains the left-hand portion of Adenovirus 5, with an artificial EagI site upstream of the E1B start site. The resulting clone, designated CN290, has a CEA-TRE inserted upstream of the E1B in a left-hand portion of adenovirus. The identity of CN290 was confirmed by restriction digest (ScaI: 2937 and 7406 bp; SmaI: 180, 783, 2628, and 6752 bp).

CN743 was generated by homologous recombination by co-transfecting 293 cells, which produces E1B, with CN290 and BHG11, which contains the wt right hand portion of Ad5. Thus, CN743 is a full-length adenoviral genome in which gene E1B is under control of a CEA-TRE.

1.C. Construction of adenovirus vectors in which expression of two adenovirus genes are each controlled by a CEA-TRE (CN742)

Briefly, a CEA-TRE fragment was inserted upstream of the E1B gene in construct CN266, which already had a CEA-TRE fragment inserted upstream of E1A. The resulting plasmid was designated CN285 and contained a left-hand portion of adenovirus with separate copies of a CEA-TRE driving expression of E1A and E1B. CN285 was recombined with a right-hand portion of adenovirus to generate CN742, which is a full-length adenovirus in which expression of both E1A and E1B is controlled by CEA-TRE.

In more detail, CN285 was constructed by amplifying the CEA-TRE inserted into the E1A region (e.g., CN266) by PCR using primers:

| | | |
|---|---|---|
| 5′ TAA CGG CCG AGC CAC CAC CCA 3′ | (39.180A, upper primer) | (SEQ ID NO:11) and |
| 5′ TAT CGG CCG GCT TGA GTT CCA GG 3′ | (39.180B, lower primer) | (SEQ ID NO:12). |

(SEQ ID NO: 12). The unique restriction site EagI was introduced by the primer pair at the ends of the PCR amplified product. The PCR product was ligated into pGEM-T Vector (Promega), and the resultant plasmid designated CN284.

The EagI CEA-TRE fragment was excised from CN284 and isolated by gel electrophoresis. The CEA-TRE fragment was ligated into CN266 which had been cut with EagI. CN266 (described above) is a left-hand portion of adenovirus in which a CEA-TRE controls expression of E1A. The resulting clone was confirmed by restriction digest (ScaI: 1682, 1732, and 7406 bp; SmaI: 783, 899 2628, and 6330 bp). The clone was designated CN285, which represents a left-hand portion of adenovirus in which both E1A and E1B are under control of separate CEA-TREs.

CN742 was generated by homologous recombination by co-transfecting 293 cells with CN285 and BHG11, which has the wt right hand portion of adenovirus. Thus, construct CN742 is a full-length adenoviral genome with genes E1A and E1B both under control of a CEA-TRE.

In short, full-length adenoviruses were constructed in which one or two adenoviral early genes were under transcriptional control of a CEA-TRE.

Table 1 lists the combinations of right end and left end Ad5 plasmids used to generate recombinant Ad5 with the desired features.

TABLE 1
Adenovirus vectors containing CEA-TRE

| Virus | Name | Left End Plasmid | Right End Plasmid |
|---|---|---|---|
| E1A-CEA | CN741 | CN266 | BHG11 |
| E1A/E1B-CEA | CN742 | CN285 | BHG11 |
| E1B-CEA | CN743 | CN290 | BHG11 |

### Example 2

### Comparative Testing of Virus Growth in vitro

Growth selectivity of CN741, CN742 and/or CN743 (full-length adenoviruses in which one or two early genes is under control of a CEA-TRE) is analyzed in plaque assays in which a single infectious particle produces a visible plaque by multiple rounds of infection and replication. Virus stocks are diluted to equal pfu/ml, then used to infect monolayers of cells for 1 hour. Comparison of normalized titres in cells that allow a CEA-TRE to function and cells that do not allow a CEA-TRE to function indicates replication preference. Cells chosen for this study are cells that allow a CEA-TRE to function, such as NCIH508, LoVo, SW 1463, MKN1, MKN28, MKN45 and cells that do not allow such function, such as HuH7 or HeLa. The inoculum is then removed and the cells are overlayed with semisolid agar containing medium and incubated at 37° C. for one week. Plaques in the monolayer are then counted and titers of infectious virus on the various cells are calculated. The data are normalized to the titer of CN702 (wild type) on 293 cells.

Full-length adenovirus CN741, in which transcription of E1A is under control of CEA-TRE, was tested in this way. Clone 46.130.8.3 was used, and CN702 (wt adenovirus) was a control. Plaques observed on cell lines were normalized to infectivity on control 293 Cells. The ratio of normalized plaques of CN741 and CN702 were compared to evaluate plaque preference in cell types. Table 2 depicts the plaque assay results. Cells examined were 293 (CEA-deficient), LoVo (CEA-producing), OVCAR (CEA-deficient), HBL100 (CEA-deficient), and HepG2 (CEA-producing). We have found that OVCAR and HBL 100 cells do not express levels of CEA detectable by ELISA, using a standard protocol with a kit purchased from Genzyme. However, while we also found that HepG2 cell do not produce CEA detectable in the ELISA test, Zhai et al. [(1990) Gastroenter. 98:470-7] showed that HepG2 cells do produce CEA, as detectable by the PAP and avidin-biotin technique.

TABLE 2
Plaque assay results of CN741 (CEA-E1A) on human cell lines

| | Normalized Plaques | Normalized Plaques | Ratio of |
|---|---|---|---|
| Cell Line | CN702 (wt) | CN741 (CEA-E1A) | CN741/CN702 |
| 293 | 1.0 | 1.0 | 1.0 |
| LoVo | 1.5 | 0.579 | 0.39 |
| OVCAR | 1.2 | 0.372 | 0.31 |
| HBL100 | 0.75 | 0.085 | 0.11 |
| HepG2 | 1.75 | 0.69 | 0.39 |

The plaque assay results in Table 2 indicate that the growth pattern of CN741 has been altered by the introduction of a CEA-TRE. In each cell line, the growth of the CN741 virus is reduced in comparison to wild-type adenovirus CN702. The ratio of CN741/CN702 in the CEA-proficient cell lines LoVo and HepG2 were similar. Importantly, there was a 4-fold reduction in the ability of CN741 to replicate in the CEA-deficient cell line HBL 10 cells. These data seem to indicate that CN741 has a greater ability (i.e., more specificity for replication) in CEA-proficient cells (LoVo and HepG2) than in CEA-deficient cells (HBL100).

Curiously, the CN741/CN702 ratio was similar in OVCAR (CEA-deficient) to that in CEA-producing cells. This suggests that replication of the CEA-E1A adenovirus relative to wt virus in OVCAR (CEA-deficient) was similar to that in CEA-producing cells. There are several possible explanations for this finding. Note that HepG2, as stated above, was determined to be CEA-deficient a CEA ELISA assay, but revealed to be CEA-proficient by the PAP and avidin-biotin technique. The ELISA method may be similarly insufficient to detect low levels of CEA present in OVCAR. Alternatively, it is possible that OVCAR cells also produce CEA, but the protein is expressed too transiently or too quickly degraded to be detectable by ELISA, yet is somehow able to allow activation of transcription of a CEA-TRE and replication of CN741.

### Example 3

### Testing Cytotoxic Ability of Adenovirus Vectors on LoVo Tumor Xenografts

An especially useful objective in the development of CEA-specific adenoviral vectors is to treat patients with CEA-producing tumors, such as hepatocellular carcinoma. An initial indicator of the feasibility is to test the vector(s) for cytotoxic activity against LoVo tumor xenografts grown subcutaneously in Balb/c nu/nu mice. Mice are given s.c. injections with 1×10⁷ LoVo carcinoma cells in PBS. Tumor cells can be tested for CEA production by assaying for CEA in serum using standard assays (for example, ELISA).

For this experiment, test adenovirus vectors are introduced into the mice either by direct intratumoral, intravenous, or intraperitoneal injection of approximately 10⁸ pfu of virus (if administered as a packaged virus) in 0.1 ml PBS and 10% glycerol or intravenously via the tail vein. If administered as a polynucleotide construct (i.e., not packaged into virus), 0.1 μg to 100 μg or more can be administered. Tumor sizes are measured and, in some experiments, blood samples are taken weekly. The effect of intratumoral injection of the adenoviral vector (such as CN733) on tumor size and serum CEA levels is compared to sham treatment.

While it is highly possible that a therapeutic based on the viruses described here would be given intralesionally (i.e., direct injection), it would also be desirable to determine if intravenous (IV) administration of adenovirus vector can affect tumor growth. If so, then it is conceivable that the virus could be used to treat metastatic tumor deposits inaccessible to direct injection. For this experiment, groups of three to five mice bearing LoVo tumors are inoculated with 10⁸ pfu of an adenoviral vector by tail vein injection, or with buffer used to carry the virus as a negative control. The effect of IV injection of the adenoviral vector on tumor size and serum CEA levels is compared to sham treatment.

### Example 4

### Testing a CEA-TRE Using a Reporter Gene Construct

A CEA-TRE can be tested in a reporter gene assay. Briefly, an adenoviral vector is constructed in which the reporter gene is under transcriptional control of the CEA-TRE. Reporter genes have been disclosed above, and can be inserted into an adenovirus and placed under control of a CEA-TRE. Methods for such construction are known in the art or are disclosed herein.

The CEA-TRE to be tested may have mutations such as deletions or insertions within binding sites known to be important in CEA-TRE activity, or base substitutions in these sites themselves.

Cells are transformed with the reporter gene construct using, for example, cationic lipids (i.e., lipofectin). After about 48 hours of culture, cells are assayed for reporter gene activity. A plasmid that lacks the CEA-TRE serves as a negative control.

A comparison of reporter gene activity in CEA-producing cells and non-CEA-producing cells, using as a control an adenovirus with a non-cell specific promoter (e.g., CMV) controlling reporter gene expression, can indicate the efficacy of a putative CEA-TRE in mediating cell-specific expression.

4.A. CEA-TRE controlling expression of luciferase

A plasmid was constructed in which a reporter gene, luciferase, was placed under transcriptional control of a CEA-TRE. This construct was found to mediate cell-specific transcription of the reporter in cells that allow a CEA-TRE to function.

A CEA-TRE was excised with PinAI from CN266 (described above), end-filled with the DNA polymerase I Klenow fragment, and blunt-end ligated into the pGL3-luc luciferase plasmid (Promega), which had been digested with SmaI. Both positive orientation (+CEA-luc) and negative orientation (−CEA-luc) clones were obtained. In the first, the CEA-TRE is operably linked to the luciferase gene; in the second, the CEA-TRE is present but not operably linked to the luciferase gene.

CEA-luciferase constructs were transfected into CEA-producing cells (LoVo) and CEA-deficient cells (293) with Lipofectin. Luciferase expression in each cell line was measured as described by standard protocols. The transfection efficiency of each cell line was determined by co-transfecting in a CMV-β-Gal construct and measuring β-galactosidase activity. 293 cells displayed a 2-fold greater transfection efficiency compared to LoVo cells, based on a comparison of β-galactosidase produced. This factor is used to normalize transfection efficiency for CEA-luciferase constructs between cell lines.

TABLE 3
Transfection of CEA-TRE luciferase plasmid info human cells

| Cell line | +CEA-luciferase plasmid | −CEA-luciferase plasmid |
|---|---|---|
| 293 | 13.8 | 2.6 |
| LoVo | 97.2 | 1.9 |

As shown in Table 3, the plasmid with the positive orientation of a CEA-TRE driving expression of the luciferase reporter gene demonstrated a 7-fold increase in luciferase expression in LoVo cells over 293 cells. Furthermore, the plasmid in which a CEA-TRE is present but does not drive expression of luciferase demonstrated only basal levels of luciferase expression.

The CEA-TRE used in this experiment is identical to that used in construct CN741, which, as described above, demonstrated a 4-fold increase in specificity in the plaque assay when comparing 293 and HBL-100 cells.

Therefore, the data shown in this Example and Example 2 show that a CEA-TRE is capable of mediating transcription and control of adenoviral replication specific to cells that allow a CEA-TRE to function, such as CEA-expressing cells.

### Example 5

### Construction of an Adenoviral Vector Containing the Coding Region for the Adenovirus Death Protein (ADP) Under Control of a CEA-TRE

An adenovirus in which the ADP gene is under control of a CEA-TRE can be constructed as described below. ADP is encoded within the E3 region and naturally under control of the major late promoter (MLP). The gene appears to code for a protein (ADP) that is important in expediting host cell lysis. Tollefson et al. (1996) J. Virol. 70(4):2296; Tollefson et al. (1992) J. Virol. 66(6):3633. Thus, adenoviral vectors containing the ADP gene may render the adenoviral vector more potent, making possible more effective treatment and/or a lower dosage requirement.

The ADP coding sequence from Ad2 can introduced into Ad5 in the E3 region (which is often deleted in the constructs; see Example 1), as follows.

An ADP cassette is constructed using overlap PCR. The Y leader, an important sequence for correct expression of some late genes, is PCR amplified using primers:

| | |
|---|---|
| 5′ GCCTTAATTAAAAGCAAACCTCACCTCCG. . .Ad2 28287bp | (37.124.1) (SEQ ID NO:13); and |
| 5′ GTGGAACAAAAGGTGATTAAAAAATCCCAG. . .Ad2 28622bp | (37.146.1) (SEQ ID NO:14). |

The ADP coding region is PCR amplified using primers

| | |
|---|---|
| 5′ CACCTTTTGTTCCACCGCTCTGCTTATTAC. . .Ad2 29195bp | (37.124.3) (SEQ ID NO:15) and |
| 5′ GGCTTAATTAACTGTGAAAGGTGGGAGC. . .Ad2 29872bp | (37.124.4) (SEQ ID NO:16). |

The two fragments were annealed and the overlap product was PCR amplified using primers 37.124.1 and 37.124.4. The ends of the product were polished with Klenow fragment and ligated to BamHI cut pGEM-72(+) (Promega, Madison, Wis.) to produce CN241. The ADP cassette was excised by digesting CN241 with PacI restriction endonuclease and ligated with two vectors, CN247 and CN248, generating plasmids CN252 and CN270, respectively.

CN247 contains a unique PacI site in the E3 region and was constructed as follows. A plasmid containing the full length Ad5 genome, TG3602 (Transgene, France), was digested with BamHI and religated to yield CN221. The backbone of this plasmid (outside of the Ad5 sequence) contained a PacI site that needed to be removed to enable further manipulations. This was effected by digesting CN221 with PacI and polishing the ends with T4 DNA polymerase, resulting in CN246. CN246 was digested with AscI and AvrII (to remove intact E3 region). This fragment was replaced by a similarly cut fragment derived from BHG11. The resulting plasmid, CN247, lacks the E3 region and has a PacI site suitable for insertion of the ADP cassette fragment (described above). Ligation of CN247 with the ADP cassette generated CN252.

CN248 (a construct that would allow introduction of an ADP cassette into a Ad that also contains a deletion/substitution in the E4 region) was made as follows. The E4 region was deleted by digesting CN108, a construct that contains right hand end Ad5 sequence from the unique EcoRI site in the E3 region, with AvrII and AfIII. The only E4 ORF necessary for viral replication, ORF 6, was reintroduced by PCR amplifying the ORF with primers,

| | |
|---|---|
| 33.81.1 (Ad5 33096): | |
| GCAGCTCACTTAAGTTCATGTCG | (SEQ ID NO:17) |
| 33.81.2 (Ad5 34084): | |
| TCAGCCTAGGAAATATGACTACGTCCG | (SEQ ID NO:18) |

The resulting plasmid is CN203. CN203 was digested with EcoRI and ligated to CN209, a shuttle plasmid, to generate CN208. In the final cloning step, CN208 was digested with AscI and AvrII and ligated to similarly cut E4 deletion/substitution with the ADP cassette.

Thus, both CN252 and CN270 are adenoviral derivatives containing the ADP and lacking the E3 gene. In addition, CN270 lacks some sequence in the E4 region as previously described. Full-length adenoviral vectors are obtained via in vitro ligation of (1) appropriately prepared viral DNA digested with BamHI and (2) CN252 or CN257 also digested with BamHI. The ligation product is used to transfect 293 cells. Plaque assays are performed as described above.

CN252 and CN270 can also be modified by insertion of a CEA-TRE fragment to place the ADP gene under control of CEA-TRE.

### Example 6

### Characterization of an E3 Deleted Adenovirus, CN751, that Contains the Adenovirus Death Protein Gene

An adenovirus comprising an adenovirus death protein, CN751, was constructed to test whether such a construct may be more effective for cytotoxicity. The adenovirus death protein (AD P), an 11.6-kDa Asn-glycosylated integral membrane peptide expressed at high levels late in infection, migrates to the nuclear membrane of infected cells and affects efficient lysis of the host. The Adenovirus 5 (Ad5) E3 region expresses the adp gene.

Construction of CN751

CN751 was constructed in two parts. First, an E3 deleted platform plasmid that contains Ad5 sequence 3′ from the BamHI site at 21562 bp was generated. The Ad2 adp was engineered into the remainder of the E3 region of this plasmid to yield CN252 (this cloning has been previously described). To construct the second part, the 5′ Ad5 sequence necessary for CN751 was obtained by digesting purified CN702 DNA with EcoRI and isolating the left hand fragment by gel extraction. After digesting CN252 with EcoRI, the left hand fragment of CN702 and CN252 were ligated. 293 cells were transfected with this ligation mixture by lipofection transfection and incubated at 37° C. Ten days later, the cells were harvested, freeze-thawed three times, and the supernatant was plaqued on 293 monolayers. Individual plaques were picked and used to infect monolayers of 293 cells to grow enough virus to test. After several days, plate lysates were screened using a polymerase chain reaction (PCR) based assay to detect candidate viruses. One of the plaques that scored positive was designated CN751.

Structural Characterization of CN751

The structure of CN751 was confirmed by two methods. First, primers 37.124.1 (5′ gccttaattaaaagcaaacctcacctccg Ad2 28287bp) and 37.124.4 (5′ggcttaattaactgtgaaaggtgggctgc Ad2 29872bp) were used to screen candidate viruses by PCR to detect the presence of the adp cassette. CN751 produced an extension fragment consistent with the expected product (1065 bp). Second, CN751 was analyzed by Southern blot. Viral DNA was purified, digested with PacI, SacI, and AccI/XhoI, and probed with a sequence homologous to the ADP coding region. The structure of CN751 matched the expected pattern.

In Vitro Characterization of CN751

Two experiments were conducted to examine the cytotoxicity and virus yield of CN751. In the first study, CN751's cytotoxicity was evaluated in LNCaP cells by measuring the accumulation of a cytosolic enzyme, lactate dehydrogenase (LDH), in the supernatant over several days. The level of extracellular LDH correlates with the extent of cell lysis. Healthy cells release very little, if any, enzyme, whereas dead cells release large quantities. LDH was chosen as a marker because it is a stable protein that can be readily detected by a simple protocol. CN751's ability to cause cell death was compared to that of CN702, a vector lacking the ADP gene, and Rec700, a vector containing the ADP gene.

Monolayers of LNCaP cells were infected at an MOI of one with either CN702, Rec700 (adp+control), or CN751 and then seeded in 96 well dishes. Samples were harvested once a day from one day after infection to five days after infection and scored using Promega's Cytotox 96 kit. This assay uses a coupled enzymatic reaction which converts a tetrazolium salt to a red formazan product that can be determined in a plate reader at 490 nm.

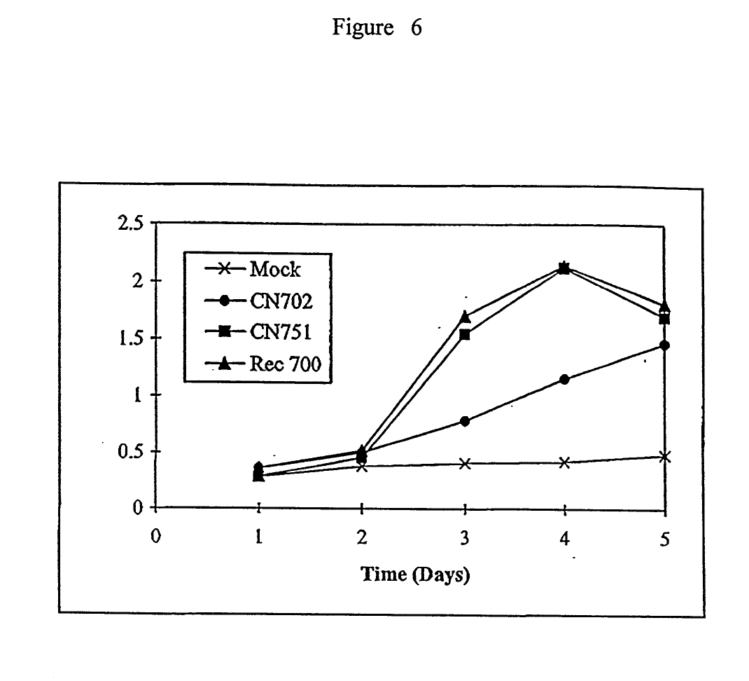

Since the absorbance of a sample corresponds to the level of LDH released from infected cells, a plot of how a sample's absorbance changes with time describes how efficiently the viruses studied induce cell lysis (FIG. 6). Each data point represents the average of sixteen separate samples. The results suggest that CN751 kills cells more efficiently than the adp− control, CN702, and similarly to the adp+ control, Rec700. The concentration of LDH in the supernatant increases rapidly from two days and reaches a maximum at four days in wells infected with CN751. In contrast, LDH concentration in the supernatant of CN702 infected cells begins to rise slowly at two days and continues until the conclusion of the experiment. Significantly, the amount of LDH released from CN751 infected cells at three days is two times that released from CN702 infected cells. In sum, the virus yield data demonstrate that adenoviral vectors with the ADP gene release more virus.

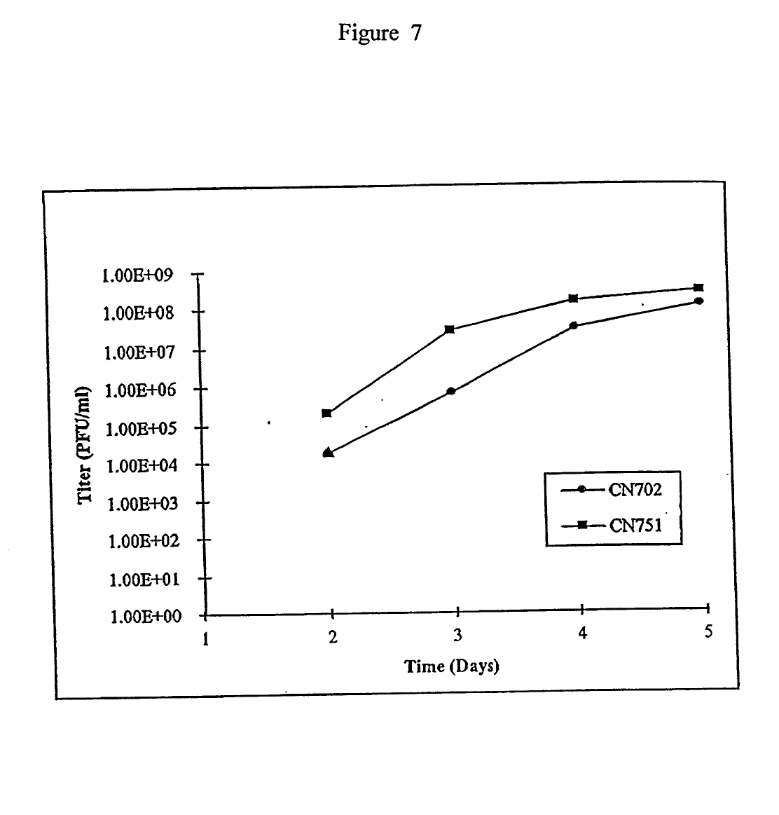

Not only is it important for Ad vectors to kill cells efficiently, they must also be able to shed progeny that can infect other cancer cells. Viral vectors that can shed large amounts of virus might be better therapeutics than those that shed only small amounts. A virus yield assay was undertaken to evaluate whether CN751 can induce the efficient release of its progeny from the infected cell. A549 cells were infected at an MOI of five. Supernatant was harvested at various times after infection and titered on 293 cells to determine the virus yield (FIG. 7). The data suggest that cells infected with CN751 shed virus more efficiently than those infected with CN702. At forty-eight hours post infection, CN751 infected cells released ten times more virus than CN702 infected. At seventy-two hours post infection, CN751 infected cells released forty times more virus. The data demonstrate that adenoviral vectors with the ADP gene kill cells more efficiently than adenoviral vectors that lack the ADP gene.

In vivo characterization of CN751

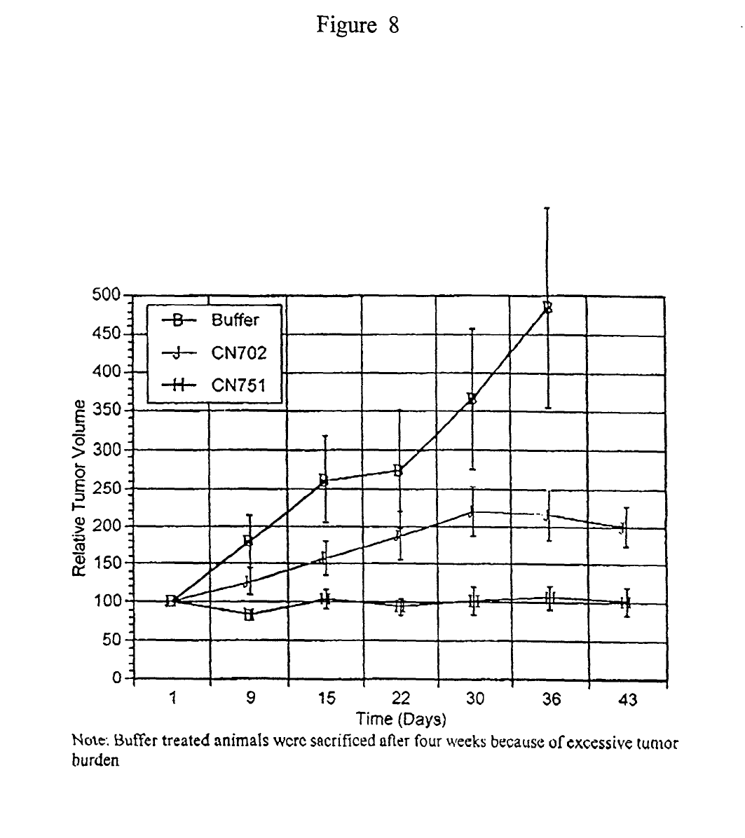

LNCaP nude mouse xenografts were challenged with a single intratumoral dose (1×10⁴ particles/mm³ tumor) of either CN751, a vector containing the ADP gene, or CN702, a vector lacking the gene. A third group of tumors was treated with buffer alone. The tumors were monitored weekly for six weeks and their relative volume was graphed against time. The results are shown in FIG. 8. Error bars represent the standard error for each sample group. The initial average tumor volume for CN751 treated animals (n=14) was 320 mm³ for CN702 treated (n=14), and 343 mm³ for buffer treated (n=8). The data suggest that CN751 kills tumor cells more effectively than CN702. On average, tumors challenged with CN751 remained the same size throughout the course of the experiments while nine out of fourteen tumors (64%) regressed. Those treated with CN702 doubled in size. Buffer treated tumors grew to nearly five times their initial volume. The Students T-test indicates that the difference in tumor size between CN751 and CN702 treated tumors was statistically significant from day 7 (p=0.016) through the end of the experiment (p=0.003).

Although the foregoing invention has been described in some detail by way of illustration and example for purposes of clarity of understanding, it will be apparent to those skilled in the art that certain changes and modifications can be practiced. Therefore, the description and examples should not be construed as limiting the scope of the invention, which is delineated by the appended claims.

What is claimed is:

1. An adenovirus vector comprising an adenovirus gene under transcriptional control of a carcinoembryonic antigen transcriptional regulatory element (CEA-TRE).

2. The adenovirus vector of claim 1, wherein the adenovirus gene is essential for viral replication.

3. The adenovirus vector of claim 2, wherein the adenovirus gene is an early gene.

4. The adenovirus of claim 2, wherein the adenovirus gene is a late gene.

5. The adenovirus vector of claim 3, wherein the adenovirus early gene is E1A.

6. The adenovirus vector of claim 2 wherein the adenovirus early gene is E1B.

7. The adenovirus vector of claim 1, wherein the adenovirus gene is the adenovirus death protein gene (ADP).

8. The adenovirus vector of claim 1, wherein the CEA-TRE comprises an enhancer from a carcinoembryonic antigen gene.

9. The adenovirus vector of claim 1, wherein the CEA-TRE comprises a promoter from a carcinoembryonic antigen gene.

10. The adenovirus vector of claim 1, wherein the CEA-TRE comprises a promoter from a carcinoembryonic antigen gene and an enhancer from a carcinoembryonic antigen gene.

11. The adenovirus vector of claim 1, wherein the CEA-TRE comprises the nucleotides about 313 to about 472 of SEQ ID NO: 1.

12. The adenovirus vector of claim 1, wherein the CEA-TRE comprises the nucleotides about 104 to about 472 of SEQ ID NO:1.

13. The adenovirus vector of claim 1, wherein the CEA-TRE comprises the sequence of SEQ ID NO: 1.

14. A composition comprising an adenovirus of claim 1.

15. A composition of claim 14, further comprising a pharmaceutically acceptable excipient.

16. The adenovirus vector of claim 1, further comprising at least one additional adenovirus gene under transcriptional control of at least one additional CEA-TRE.

17. A composition comprising an adenovirus of claim 16.

18. The composition of claim 17, further comprising a pharmaceutically acceptable excipient.

19. An adenovirus vector of claim 1, further comprising a heterologous gene under transcriptional control of a carcinoembryonic antigen transcriptional regulatory element (CEA-TRE).

20. The vector of claim 19, wherein the heterologous gene is a reporter gene.

21. The vector of claim 19, wherein the heterologous gene is conditionally required for cell survival.

22. A host cell transformed with an adenovirus vector of claim 1.

23. A host cell transformed with an adenovirus vector of claim 16.

24. A method of detecting cells that allow a CEA-TRE to function in a biological sample comprising the steps of:
contacting a biological sample with an adenovirus vector of claim 1, under conditions suitable for CEA-TRE-mediated gene expression in cells that allow a CEA-TRE to function; and
determining if CEA-TRE mediates gene expression in the biological sample,
wherein CEA-TRE-mediated gene expression is indicative of the presence of cells that allow a CEA-TRE to function.

25. A method of propagating adenovirus specific for cells that allow a CEA-TRE to function, said method comprising:
combining an adenovirus according to claim 1 with cells that allow a CEA-TRE to function,
whereby said adenovirus is propagated.

26. A method of propagating an adenovirus specific for cells that allow a CEA-TRE to function, said method comprising:
combining an adenovirus according to claim 16 with cells that allow a CEA-TRE to function,
whereby said adenovirus is propagated.

27. A method for modifying the genotype of a target cell, said method comprising contacting a cell that allow a CEA-TRE to function with an adenovirus vector of claim 1, wherein the vector enters the cell.

28. A method for modifying the genotype of a target cell, said method comprising contacting a cell that allow a CEA-TRE to function with an adenovirus vector of claim 16, wherein the vector enters the cell.

29. A method for conferring selective cytotoxicity on a target cell, said method comprising contacting a cell that allow a CEA-TRE to function with an adenovirus vector of claim 1, wherein the vector enters the cell.

30. A method for conferring selective toxicity on a target cell, said method comprising contacting a cell that allows a CEA-TRE to function with an adenovirus vector of claim 16, wherein the vector enters the cell.

31. A method of treating a CEA-associated tumor in an individual, comprising the step of administering to the individual an effective amount of an adenovirus vector of claim 2.

32. The method of claim 31, wherein the adenovirus gene is an early gene.
